(19) United States
(12) Patent Application Publication
(10) Pub. No.: US 2021/0164001 A1
(43) Pub. Date: Jun. 3, 2021

(54) CAPTURING AND CONVERTING CO2 INTO BIODEGRADABLE BIOPLASTIC

(71) Applicant: KING ABDULLAH UNIVERSITY OF SCIENCE AND TECHNOLOGY, Thuwal (SA)

(72) Inventors: Israa Salem AL ROWAIHI, Thuwal (SA); Stefan Wolfgang GRÖTZINGER, Thuwal (SA); Ram KARAN, Thuwal (SA); Jörg EPPINGER, Thuwal (SA); Stefan Theodor AROLD, Thuwal (SA)

(73) Assignee: King Abdullah University of Science and Technology, Thuwal (SA)

(21) Appl. No.: 17/044,982

(22) PCT Filed: Apr. 3, 2019
(86) PCT No.: PCT/IB2019/052735

§ 371 Date: Oct. 2, 2020

Related U.S. Application Data
(60) Provisional application No. 62/770,265, filed on Nov. 21, 2018.
(60) Provisional application No. 62/651,978, filed on Apr. 3, 2018.

(51) Int. Cl. C12P 7/62; C12M 1/00; C12M 1/34; C12N 1/20

(52) U.S. Cl. CPC C12P 7/625; C12N 1/20; C12M 41/40; C12M 23/58

(57) ABSTRACT

Embodiments of the present disclosure describe methods, systems, and compositions for the production of bioplastic from a gaseous substrate containing a carbon source by a two-stage fermentation process. Stage I includes introducing a gaseous substrate comprising CO₂ and H₂ to a primary bioreactor containing a culture of an acetogenic microorganism, anaerobically fermenting the gaseous substrate under elevated pressure to produce an acid-containing culture medium, and stage II includes aerobically fermenting the acid of the culture medium in a secondary bioreactor containing a culture of a PHA-producing microorganism to produce a polyhydroxyalkanoate (PHA). The culture medium can be suitable for both the first and second stages and enable the primary and secondary bioreactors to be connected in a sustainable continuous system.

### BACKGROUND

Carbon dioxide (CO₂) is an abundant, nontoxic, recyclable, and relatively pure by-product of many industrial processes. Moreover, CO₂ is an economically and environmentally favored raw material for bioprocessing methods that are designed for sustainability and waste management. The application of microorganisms to CO₂ fermentation has numerous advantages over alternative thermo-chemical approaches (e.g., the Fischer-Tropsch process, FTP), such as lower temperatures and the high specificity of the enzymes involved in the microbial pathway. Additionally, microorganisms exhibit higher tolerance to poisoning by tars, sulfur, and chlorine than do inorganic catalysts, and their low processing temperature offers significant energy and cost savings.

Microbial CO₂ fermentation can be performed by acetogens, a class of anaerobic microorganisms that synthesize acetyl-CoA and cellular carbon from CO₂ using the Wood-Ljungdahl pathway (WLP) (see FIG. 2) to produce acetic acid. Acetic acid is an attractive product of bioprocesses that capture CO₂ because the autocatalytic nature of microbes in liquid media is advantageous for its production. Acetic acid has a broad spectrum of applications as a solvent and as a key raw material for many products, including polymers, paints, adhesives, paper coatings, and textile treatment. These applications result in a global acetic acid demand of 6.5 million metric tons per year (Mt/a), of which only about 1.5 Mt/a come from recycled sources. The efficient recovery of acetic acid remains challenging and has received considerable attention recently.

Two-stage biological processes that transfer gas to liquid (Bio-GTL) are a promising technology that combines two microbial processes: (i) the metabolism of gaseous substrates (e.g., CO₂); and (ii) the synthesis of bioproducts. Bio-GTL processes are suitable to produce acetic acid from CO₂ fermentation and convert it into a more valuable product. A Bio-GLT process that bypasses the expensive recovery process of acetate in aqueous phase would reduce production costs.

Bio-GTL processes can be used for the production of polyhydroxyalkanoates (PHAs). PHAs are a class of microbially produced biopolymers with potential to be used in many applications, including packaging, biomedicine and agriculture. Due to higher production costs of PHA produced from more expensive carbon sources, a Bio-GTL process using CO₂ and acetate could provide a cheaper and sustainable process for PHA production.

A Bio-GTL process producing PHAs from CO₂ was reported by Lagoa-Costa et al. (Bioresource Technology 239: 244-249 (2017)). In the Lagoa-Costa et al. process, the first stage included anaerobic syngas fermentation carried out by Clostridium autoethanogenum to produce ethanol and acetic acid, followed by a second stage that converted the acetic acid into PHA with a mixed microbial aerobic culture.

There is a need to develop Bio-GTL processes with improved efficiency and sustainability over existing carbon capture and conversion processes. For example, there is a need for improved methods, compositions, and systems to minimize production costs, increase gas-to-liquid mass transfer, decrease process time, and otherwise improve the cost-effectiveness of microbially-produced acetic acid and bioplastics.

### SUMMARY

In general, embodiments of the present disclosure describe methods, systems, and compositions for capturing carbon from a gaseous substrate using a two-stage process.

Accordingly, the present disclosure features a method for producing bioplastic from a gaseous substrate comprising a carbon source, the method comprising: introducing a gaseous substrate comprising CO₂ and H₂ into a primary bioreactor comprising a culture of an acetogenic microorganism in a culture medium; anaerobically fermenting the gaseous substrate in the primary bioreactor at a pressure of at least 2 bar absolute to produce an acid-containing culture medium; introducing at least a portion of the acid-containing culture medium to a secondary bioreactor; inoculating the secondary bioreactor with a culture of a polyhydroxyalkanoate (PHA)-producing microorganism, and aerobically fermenting the acid in the culture medium to produce a PHA. In some embodiments of the method, the ratio of H₂:CO₂ in the gaseous substrate is within a range of about 75:25 to about 95:5. In some cases, the partial pressure of H₂ in the primary bioreactor is within a range of 1.7 bar to about 5 bar absolute. In one or more of the embodiments above, after consumption of a portion of the gaseous substrate by the acetogenic microorganism, the method further comprises introducing sufficient additional gaseous substrate to maintain the pressure in the primary bioreactor at or above 2 bar absolute. In one or more embodiments above, the acetogenic microorganism is selected from the group consisting of Acetobacterium, Clostridium, Eubacterium, Bacteroides, Sporomusa, Acetogenium, and Morera. In one or more of the embodiments described above, the PHA-producing microorganism is selected from the group consisting of Cupriavidus necator H16, Pseudomonas putida mt-2, Bacillus spp. type strains, Corynebacterium glutamicum, Corynebacterium hydrocarboxydans, Nocardia lucida, and Rhodococcus sp. In one or more of the embodiments described above, the method further comprises adjusting the pH of the portion of acid-containing culture medium in the secondary bioreactor before inoculating with the culture of the PHA-producing microorganism. In some cases, the pH is adjusted within a range of about 7 to 8. In one or more embodiments of the method described above, introducing the portion of the acid-containing medium is performed when the concentration of the acid is within the range of about 2.5 g L⁻¹ to about 20 g L⁻¹ culture medium in the primary bioreactor. In one or more of the embodiments described above, introducing the portion of the acid-containing culture medium comprises separating the acetogenic microorganism from the acid-containing culture medium. In one or more embodiments described above, the method further comprises obtaining the gaseous substrate from a renewable resource. In some cases, the method comprises obtaining H₂ by photovoltaic electrolysis, photoelectrochemical water splitting, or biomass gasification. In one or more of the embodiments above, the method further comprises separating the PHA-producing microorganism from the secondary bioreactor to produce a separated culture medium and introducing the separated culture medium into the primary bioreactor. In one or more of the embodiments described above, the method further comprises extracting the PHA from the PHA-producing microorganism. In one or more embodiments, the method further comprises oxygenating the culture medium in the secondary bioreactor. In some cases, oxygenating comprises introducing oxygen obtained from a renewable resource into the secondary bioreactor. In one or more of the embodiments described above, the primary and secondary bioreactors are connected.

The present disclosure further features a system for producing bioplastic from a gaseous substrate comprising a carbon source, the system comprising: a primary bioreactor and a secondary bioreactor, wherein the primary bioreactor is configured for receiving a gaseous substrate comprising CO₂ and H₂ and enabling anaerobic fermentation of the gaseous substrate at a pressure of at least 2 bar absolute for the production of an acid-containing culture medium by an acetogenic microorganism; wherein the secondary bioreactor is configured for receiving at least a portion of the acid-containing culture medium from the primary bioreactor and enabling aerobic fermentation of the acid for the production of a polyhydroxyalkanoate (PHA) by an PHA-producing microorganism. In some cases, the primary bioreactor comprises a pressure regulating system enabling the pressure to be maintained during anaerobic fermentation. In one or more embodiments of the system, the primary bioreactor is coupled to the secondary bioreactor by a conduit. In one or more embodiments of the system, the primary bioreactor comprises a gas dosing system configured to deliver the gaseous substrate to the primary bioreactor only when the pressure drops below 2 bar absolute. In one or more of the embodiments above, the secondary bioreactor includes a pH control system for adjusting the culture medium for growth of the PHA-producing microorganism and the accumulation of PHA.

The present disclosure also features a culture medium for production of a polyhydroxyalkanoate (PHA) by a PHA-producing microorganism, the culture medium made by a process comprising: introducing a gaseous substrate comprising CO₂ and H₂ into a primary bioreactor comprising a culture of an acetogenic microorganism in an anaerobic culture medium; anaerobically fermenting the gaseous substrate in the primary bioreactor at a pressure of at least 2 bar absolute to produce an acid-containing culture medium; introducing at least a portion of the acid-containing culture medium to a secondary bioreactor; and adjusting the pH of the acid-containing culture medium for growth of a PHA-producing microorganism and production of a PHA by aerobic fermentation. In some cases, the anaerobic culture medium includes, in water, about 2-6 g L⁻¹ yeast extract, about 1 g L⁻¹ reducing agent, about 5-10 g L⁻¹ bicarbonate, a mineral or trace element, a chelator, a B vitamin or B vitamin precursor, and a buffering agent. The reducing agent can be selected from the group consisting of L-cysteine, thioglycolate, sodium dithionite, dithiothreitol, iron(II) sulfide and sodium sulfide, and combinations thereof. The mineral or trace element can be selected from the group consisting of magnesium, nitrogen, calcium, sodium, manganese, cobalt, zinc, iron, nickel, aluminum, copper, boron, molybdenum, selenium, and tungsten or salts/chelates and combinations thereof. The chelator can be selected from the group consisting of ethylenediaminetetraacetic acid and nitrilotriacetic acid. The B vitamin or B vitamin precursor can be selected from the group consisting of pyridoxine, pantothenate, lipoic acid, nicotinic acid, p-aminobenzoic acid, riboflavin, thiamine, biotin, folic acid, and cobalamin, or combinations thereof. The pH of the anaerobic culture medium can be within the range of 7-8.2. In one or more embodiments of the process for preparing the culture medium, the pH is adjusted to be slightly alkaline. In one or more embodiments above, the culture medium is effective for the growth of the PHA-producing organism and accumulation of PHA of greater than about 24% PHA weight/cell dry weight within about 10-18 hours of growth.

The methods, systems, and compositions, described in the present disclosure can be utilized as a Bio-GTL process that exhibits improved efficiency relative to processes that capture carbon from gas under atmospheric pressure or which feed the gaseous substrate at a continuous flow rate, processes that require more than one culture medium, or processes that are limited to batch-fermentation or open-loop systems. The methods, systems, and compositions described in the present disclosure can reduce production cost and increase gas-to-liquid mass transfer. For example, using a method, system, or culture medium in the present disclosure can improve the cost-effectiveness of microbially-produced acetate and bioplastics by enabling a closed-loop system that uses the culture medium of stage I to be used in stage II and optionally recycled to be used again. Additional advantages, such as a reduction in gas waste and conversion time, are realized by performing pressurized fermentation using gas dosing to rebuild pressure as opposed to processes using a constant flow rate to provide the gaseous substrate during anaerobic fermentation.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale, like numerals describe substantially similar components throughout the several views. Like numerals having different letter suffixes represent different instances of substantially similar components. The drawings illustrate by way of example, but not by way of limitation, various embodiments discussed in the present document.

Reference is made to illustrative embodiments that are depicted in the figures, in which:

FIG. 1 is a flow diagram of a method of capturing and converting gaseous CO₂ into a biodegradable bioplastic (PHB), with acetate as an intermediate product, according to one or more embodiments of the present disclosure.

FIG. 2 is a schematic overview of the Wood-Ljungdahl pathway (WLP), the central metabolic pathway of A. woodii, an acetogen utilized according to one or more embodiments of the present disclosure. (Abbreviations: HDCR: hydrogen-dependent carbon dioxide reductase. THF: Tetrahydrofolate. CoFeSP: corrinoid iron-sulfur protein. CODH/ACS: CO Dehydrogenase/Acetyl-CoA Synthase. Pta: phosphotransacetylase. Ack: acetate kinase. Rnf: a novel ion-motive electron transport complex in prokaryotes. Fd: ferredoxin. Hyd: hydrogenases).

FIG. 3 is a schematic overview of a strategy for developing a two-stage Bio-GTL process, according to one or more embodiments of the present disclosure. In stage I, conversion of CO₂ and H₂ to acetate includes fermentation of the gas mixture (CO₂:H₂) using A. woodii in a high-pressure stirred-tank reactor to increase the gas-to-liquid mass transfer. In stage II, the growth of R. eutropha on acetate includes determining the effect of different concentrations of acetate on growth and acetate uptake is screened in a milliliter-scale setup while varying pH to determine toxic levels of acetate. Bio-GTL integrates the kinetics and thermodynamics for the reactions involved in each stage and the conditions with the highest energy efficiency for both stages using one medium for both microorganisms.

FIGS. 4(A)-(D) show the autotrophic processes of A. woodii in a high-pressure stirred-tank bioreactor under 2.0 and 5.5 bar pressure (gray lines and black lines, respectively), according to one or more embodiments of the present disclosure. (A) shows cell dry weight (CDW) (g L⁻¹), (B) shows the space-time yield, (C) shows the specific acetate formation rate (q_{acetate}); and (D) shows acetate concentration (AcOH), as a function of process time.

FIG. 5 is an HPLC chromatogram showing products of A. woodii in an anaerobic medium under the higher-pressure autotrophy, according to one or more embodiments of the present disclosure.

FIG. 6 shows metabolite production of A. woodii grown under 5.5 bar in medium ‘A3’, according to one or embodiments of the present disclosure. Acetic acid (AcOH, black triangles), formic acid (grey diamonds), pyroglutamic acid (grey square) and uracil (black x). A potential pathway for the formation of these compounds is presented in FIG. 7.

FIG. 7 is a schematic of CO₂ capture by A. woodii via the WLP, according to one or more embodiments of the present disclosure. During autotrophic acetogenesis, acetate is formed using 4H₂ and 2CO₂. With higher CO₂ concentrations in the medium, the precursor, acetyl-CoA is later converted to pyruvate, which is sequentially reduced to lactate or other compounds, depending on the availability of nitrogen. HDCR: hydrogen-dependent carbon dioxide reductase. THF: Tetrahydrofolate. CoFeSP: corrinoid iron-sulfur protein. CODH/ACS: CO Dehydrogenase/Acetyl-CoA Synthase. Pta: phosphotransacetylase. Ack: acetate kinase. Rnf: a novel ion-motive electron transport complex in prokaryotes. Fd: ferredoxin. Hyd: hydrogenases.

FIGS. 8(A)-(D) show R. eutropha H16 growth in different acetate concentrations and at different pH, according to one or more embodiments of the present disclosure. Cultures were pre-grown for 8 h, then acetate was added, and growth was continued for an additional 10 h. The change in cell density (ΔOD₆₀₀) is shown after pre-growth, between 8 h to 18 h. (A) pH 6.5 (black circles), (B) pH 7.0 (gray squares), (C) pH 7.5 (black triangles), (D) pH 8.0 (black triangles).

FIG. 9 is a schematic of a potential continuous or semi-continuous system for production of PHB by capturing CO₂ using H₂ generated by photovoltaics (PV), according to one or more embodiments of the present disclosure. In the first reactor, H₂ is produced electrochemically with PV-produced energy. Autotrophy takes place in a primary bioreactor (stage I) with the PV-produced H₂ and external CO₂ to produce acetate under high-pressure conditions using A. woodii (gray ovals). In a secondary bioreactor (stage II), the acetate is converted to PHB by R. eutropha (black ovals). The media is filtered and recycled to stage I. The two stage process mimics photosynthesis (in theory: 30%-18% renewables (e.g., harvesting solar energy), 90% CO₂ capture, 73% higher C products). R. eutropha (24 h) in acetate media from A. woodii culture (2 bar): AcOH uptake=3.0 g L⁻¹ (48 mmol L⁻¹), PHB produced=0.5 g L⁻¹ (5.8 mmol L⁻¹). Under one embodiment, the two stages are: (I) autotrophy using A. woodii to produce acetate; and (II) heterotrophy using R. eutropha H16 for conversion of acetate into biodegradable bioplastic, poly-3-hydroxybutyrate (PHB).

FIG. 10 is a schematic showing the efficiency of CO₂ capture in photosynthesis and the comparative efficiency of a Bio-GLT process, according to one or more embodiments of the present disclosure. Photosynthetic efficiency (sun to bio-product energy) overlaps the range of theoretical values. Theoretic calculated efficiency is 0.5-7.0% whereas the efficiency achieved by nature is 0.1-1.0%.

### DETAILED DESCRIPTION

The present disclosure features methods, systems, and compositions for converting CO₂ to bioplastic using a two-stage biological gas to liquid transfer process (Bio-GTL). Methods, systems, and compositions of the present disclosure can improve process time, increase carbon fixation (e.g., increase acetate production and maximum acetate formation rate) and space-time yield while minimizing gas loss relative to processes using constant flow rates of gas to feed anaerobic fermentation at atmospheric pressure and media that are unable to be used in more than one stage of a biological gas-to-liquid process.

### Definitions

The terms recited below have been defined as described below. All other terms and phrases in this disclosure shall be construed according to their ordinary meaning as understood by one of skill in the art.

The terms “a” and “an,” as used herein, are defined as one or more than one (e.g., at least one). The use of introductory phrases such as “at least one” and “one or more” in the disclosure should not be construed to imply that another element introduced by “a” or “an” is limited to embodiments containing only one such element. The use of the definite article is also non-limiting with respect to whether the element is a single element or a plurality.

As used herein, “acetogen” or “acetogenic” refers to a microorganism that generates acetate as a product of autotrophic anaerobic respiration. The term “homoacetogen” refers to a strictly anaerobic microorganism that catalyzes the formation of acetate from C₁ units, most of which are able to grow at the expense of hydrogen with CO₂ as the sole energy source.

As used herein, “autotroph” or “autotrophic” refers to an organism that is capable of using inorganic materials (e.g., carbon dioxide) to manufacture its organic nutritional requirements.

As used herein, “bioplastic” refers to a plastic derived from one or more types of renewable biomass, such as linear polyesters synthesized by microorganisms under stress conditions and accumulated in inclusion bodies (i.e., intracellular granules, AKA carbonosomes). Polyhydroxyalkanoates (PHAs) are exemplary biodegradable bioplastics.

As used herein, “bioreactor” refers to a system used for bioprocessing, which supports the growth of cells by simulating a natural biochemical environment. A bioreactor can be a fermenter for anaerobic or aerobic microbial culture. Examples of bioreactors include flasks, roller bottles, tanks, vessel or other containers. Bioreactors range in size from small, benchtop units, to large-scale systems for industrial applications. A bioreactor can be a batch-type or continuous-flow type unit. A bioreactor is not limited to the reaction vessel and includes structures permitting control over parameters such as temperature, moisture, pH, oxygen, and agitation. Examples of suitable bioreactors include a continuous stirred tank reactor, continuous flow reactor, immobilized cell reactor, bubble column reactor, and trickle bed reactor.

A first aspect of the invention of the present disclosure will now be described with reference the following clauses of which:

Clause 1—a method of producing bioplastic from a gaseous substrate comprising a carbon source, the method comprising:

introducing a gaseous substrate comprising CO₂ and H₂ into a primary bioreactor comprising a culture of an acetogenic microorganism in a culture medium;

anaerobically fermenting the gaseous substrate in the primary bioreactor at a pressure of at least 2 bar absolute to produce an acid-containing culture medium; introducing at least a portion of the acid-containing culture medium to a secondary bioreactor;

inoculating the secondary bioreactor with a culture of a polyhydroxyalkanoate (PHA)-producing microorganism, and aerobically fermenting the acid in culture medium to produce a PHA.

Clause 2—The method according to clause 1 in which the ratio of H₂:CO₂ in the gaseous substrate is within a range of about 75:25 to about 95:5.

Clause 3—The method according to clause 1 or clause 2, wherein the partial pressure of H₂ in the primary bioreactor is within a range of 1.7 bar to about 5 bar absolute.

Clause 4—The method according to any of clauses 1 to 3, wherein after consumption of a portion of the gaseous substrate by the acetogenic microorganism, the method further comprises introducing sufficient additional gaseous substrate to maintain the pressure in the primary bioreactor at or above 2 bar absolute.

Clause 5—The method according to any of clauses 1 to 4, wherein the acetogenic microorganism is selected from the group consisting of Acetobacterium, Clostridium, Eubacterium, Bacteroides, Sporomusa, Acetogenium, and Morera.

Clause 6—The method according to any of clauses 1 to 5, wherein the PHA-producing microorganism is selected from the group consisting of Cupriavidus necator H16, Pseudomonas putida mt-2, Bacillus spp. type strains, Corynebacterium glutamicum, Corynebacterium hydrocarboxydans, Nocardia lucida, and Rhodococcus sp.

Clause 7—The method of any of clauses 1 to 6, comprising adjusting the pH of the portion of acid-containing culture medium in the secondary bioreactor before inoculating with the culture of the PHA-producing microorganism.

Clause 8—The method of clause 7, wherein the pH is adjusted within a range of about 7 to about 8.

Clause 9—The method of any of clauses 1 to 8, wherein introducing the portion of the acid-containing medium is performed when the concentration of the acid in the primary bioreactor is within the range of about 2.5 g L⁻¹ to about 20 g L⁻¹ culture medium.

Clause 10—The method of any of clauses 1 to 9, wherein introducing the portion of the acid-containing culture medium comprises separating the acetogenic microorganism from the acid-containing culture medium.

Clause 11—The method of any of clauses 1 to 10, comprising obtaining the gaseous substrate from a renewable resource.

Clause 12—The method of any of clauses 1 to 11, comprising obtaining the H₂ of the gaseous substrate by photovoltaic electrolysis, photoelectrochemical water splitting, or biomass gasification.

Clause 13—The method of any of clauses 1 to 12, comprising separating the PHA-producing microorganism in the secondary bioreactor to produce a separated culture medium and introducing the separated culture medium to the primary bioreactor.

Clause 14—The method of any of clauses 1 to 13, comprising extracting the PHA from the PHA-producing microorganism.

Clause 15—The method of any of clauses 1 to 14, comprising oxygenating the culture medium in the secondary bioreactor.

Clause 16—The method of clause 15 in which oxygenating comprises introducing oxygen obtained from a renewable resource into the secondary bioreactor.

Clause 17—The method of any of clauses 1 to 16, wherein the primary and secondary bioreactors are connected.

A second aspect of the invention of the present disclosure will now be described with reference to the following clauses of which:

Clause 18—A system for producing bioplastic from a gaseous substrate comprising a carbon source, the system comprising:

a primary bioreactor and a secondary bioreactor,

wherein the primary bioreactor is configured for receiving a gaseous substrate comprising CO₂ and H₂ and enabling anaerobic fermentation of the gaseous substrate at a pressure of at least 2 bar absolute for the production of an acid-containing culture medium by an acetogenic microorganism;

wherein the secondary bioreactor is configured for receiving at least a portion of the acid-containing culture medium from the primary bioreactor and enabling aerobic fermentation of the acid for the production of a polyhydroxyalkanoate (PHA) by a PHA-producing microorganism.

Clause 19—The system of clause 18 in which the primary bioreactor comprises a pressure regulating system that enables the pressure to be maintained during anaerobic fermentation.

A third aspect of the invention of the present disclosure will now be described with reference to the following clause of which:

Clause 20—A culture medium for the production of a polyhydroxyalkanoate (PHA) by a PHA-producing microorganism, the culture medium made by a process comprising:

introducing a gaseous substrate comprising CO₂ and H₂ into a primary bioreactor comprising a culture of an acetogenic microorganism in an anaerobic culture medium;

anaerobically fermenting the gaseous substrate in the primary bioreactor at a pressure of at least 2 bar absolute to produce an acid-containing culture medium;

introducing at least a portion of the acid-containing culture medium to a secondary bioreactor; and

adjusting the pH of the acid-containing culture medium for growing a culture of a PHA-producing microorganism and producing a PHA by aerobic fermentation.

The present disclosure features a method for microbially capturing carbon in a gaseous source to produce an acid fermentation product and converting acid into one or more bioplastics. The method can be described as a biological gas-to-liquid transfer process or Bio-GTL process. The Bio-GTL process can have at least two stages. Generally, the stage I includes introducing a gaseous substrate comprising CO₂ and an electron source (H₂) to a culture of an acetogen in a liquid culture medium in a primary bioreactor. The acetogen converts the inorganic carbon to an acid by anaerobic autotrophic fermentation. The acid is excreted into the culture medium. Generally, stage II includes introducing the acid-containing culture medium to a secondary bioreactor, and inoculating the acid-containing culture medium with a culture of a PHA-producing microorganism. The PHA-producing organism produces a bioplastic by aerobic fermentation using the acid of the culture medium as a fermentation substrate.

### Stage I

The Gaseous Substrate

In one or more embodiments of the present disclosure, stage I of a Bio-GTL process includes a step of providing a gaseous substrate containing a carbon source. The carbon source is selected for the production of an acid by autotrophic fermentation. Microbial production of an acid, such as acetic acid, by autotrophic fermentation, utilizes CO₂ at the expense of hydrogen. Therefore, the gaseous substrate can include CO₂ and H₂. The gaseous substrate can be provided for acetate production as a mixture of CO₂ and H₂ gases under pressure (e.g., in a mixed-gas tank). Alternatively, the gaseous substrate may be provided from a waste gas stream containing CO₂ and H₂ collected from emissions or industrial processes. For example, a waste gas stream containing CO₂ and H₂ can be collected from an industrial process, combustion of fossil fuels such as coal, oil, and gas in power plants or automobiles, or industrial facilities producing hydrogen, ammonia, limestone or cement.

Waste gas streams rich in H₂ or rich in CO₂ may be more readily available than waste gas streams rich in both H₂ and CO₂. Accordingly, the gaseous substrate comprising H₂ and CO₂ may be provided by blending one or more gas streams. H₂-rich gas streams can be produced by a variety of processes including steam reformation of hydrocarbons or natural gas, partial oxidation of coal or hydrocarbons, electrolysis of water, or by-products from electrolytic cells (e.g., chlorine producing cells). In one or more embodiments of the present disclosure, a H₂-rich stream can be produced indirectly from a renewable resource (e.g., via electricity generated by wind power, solar power, geothermal power or biomass gasification) or directly from a renewable resource such as solar energy (a photovoltaic cell) or biomass gasification. CO₂-rich streams can be produced from natural CO₂ wells, hydrocarbon combustion (e.g., natural gas or oil) or as an industrial process by-product (e.g., from the production of ammonia, lime or phosphate). In one or more embodiments of the present disclosure, the CO₂-rich stream is a by-product of an alcohol fermentation process (e.g., syngas fermentation to produce ethanol). CO₂ can be separated from other gaseous components of a gas stream by known methods, such as introducing solvents that absorb CO₂ from flue gas after combustion, hydrocarbon gasification, water-shift reaction, and combustion of hydrocarbons in pure oxygen.

Gas streams with a high CO₂ content, but minimal or no H₂, can be blended with one or more streams comprising H₂ prior to introducing the gaseous substrate to a primary bioreactor. The primary bioreactor can be one configured to receive the gaseous substrate and accommodate a culture of an autotrophic microorganism capable of converting CO₂ to an acid (e.g., an acetogen). The stoichiometry of the CO₂ and H₂ in the blended stream can influence acid production (e.g., acetate production) efficiency and/or total carbon capture by autotrophic fermentation. In one or more embodiments of the present disclosure, the gaseous substrate can be blended to comprise H₂ and CO₂ in a ratio of about 75:25 to about 95:5, such as a ratio of about 85:15. The blended (or mixed) substrate can contain a major proportion of H₂ by volume, such as at least about 30%, at least 40%, at least 50%, at least 60%, at least 70%, or about 75% H₂ by volume. The blended (or mixed) substrate gaseous substrate can contain at least about 5% CO₂ by volume, such as at least 10%, at least 15%, at least 20%, or about 25% CO₂ by volume.

Blending can be achieved by flowing individual gas streams into a mixing chamber, such as a small vessel, or a section of pipe, which can be coupled to the primary bioreactor. The vessel or pipe, or other mixing chamber, may include a mixing device, such as baffles, arranged to promote turbulence and homogenization of the individual gas streams to obtain a gaseous substrate having the target ratio of CO₂ and H₂. Blending can be monitored by a processor adapted to monitor the composition and flow rates of the constituent gas streams and control the proportion of CO₂ and H₂.

Providing a gaseous substrate for use in the methods and systems of the present disclosure can include treating the gas to remove impurities, such as dust particles or gases that may inhibit fermentation or inhibit the production of a desired fermentation product. In one or more embodiments, the gaseous substrate may be filtered, scrubbed, or otherwise treated to reduce particulates, sulfur compounds, CO, CH₄, O₂, N₂, NH₃, or water vapor.

Autotrophic Fermentation

Autotrophic fermentation of the gaseous substrate is performed using conditions appropriate for conversion of CO₂ to acid for the production of bioplastic. In one or more embodiments described in the present disclosure, stage I includes anaerobic fermentation of the gaseous substrate by a homoacetogen to form acetate as a fermentation product. Conditions that influence the rate of conversion include pressure, temperature, composition of the gaseous substrate, inoculum level, as well as the culture medium (e.g., its composition, redox potential, concentration of dissolved gases, and pH). These conditions influence the composition of the metabolic products produced by the microorganism. In addition, metabolic products of autotrophic fermentation may feedback or forward to one or more metabolic pathways to influence production of a desired fermentation product. For example, nutrient content of the medium can result in a high rate of NADH production, excess acetyl-CoA, and ultimately production of pyruvate in addition to acetate. Optimal conditions for producing a specific product can be determined empirically using the guidance provided in the present disclosure. For example, the optimum temperature for growth of the bacteria, and highest production rate of acetate can be determined by running the bioreactor, or a separate fermenter (e.g., shaking flasks comprising a culture of acetogenic microorganisms, or benchtop single-use bioreactor), at a range of different temperature points. In one or more embodiments described herein, the primary bioreactor temperature is maintained at about 30° C.

In one or more embodiments of the present disclosure, the amount of gaseous substrate provided during autotrophic fermentation is controlled to minimize loss of gas and thereby increase gas-to-liquid conversion efficiency. In the methods described herein, anaerobic fermentation is performed at a pressure higher than atmospheric pressure. Operating at increased pressure allows a significant increase in the rate of transfer of CO₂ and H₂ from the gas phase to the liquid phase. The pressure can be initially set to a pressure greater than or equal to 2 bar absolute. In some cases, the pressure is set within a range of ≥2 and less than 5.5 bar absolute. During fermentation, consumption of the gaseous substrate results in a loss of pressure. The primary bioreactor can be configured or adapted with a pressure regulating system configured to monitor consumption of the gaseous substrate and to periodically rebuild the pressure to the initial pressure by flowing additional gaseous substrate into the reaction vessel after at least a portion of the gaseous substrate is consumed. Alternatively, anaerobic fermentation can use a combination of a substantially continuous stream of gaseous substrate (i.e., a constant flow rate), a semi-continuous stream of gaseous substrate to avoid a drop below 2 bar absolute (e.g., volumetric regulation), and doses of gaseous substrate to rebuild the pressure after the pressure drops below 2 bar absolute during fermentation. A suitable pressure regulation strategy can be determined empirically based on the efficiency of the conversion of the substrate to the acid. The optimum pressure conditions will depend partly on the particular microorganism used and can be determined using the guidance provided in this disclosure.

The optimum pressure conditions can also depend on the agitation rate. The primary bioreactor can be agitated by shaking (e.g., flask) or stirring (e.g., stirred reactor). In one or more embodiments, the primary bioreactor is shaken or stirred within a range of rotations speeds, such as from about 100 to about 1200 rpm or about 150 to about 500 rpm.

In stage I, the gaseous substrate is introduced to a culture of an acetogenic microorganism that uses H₂ and CO₂ to produce an acid under anoxic conditions (e.g., a homoacetogen). The culture can be a mixed culture of two or more acetogenic microorganisms, or a culture of a single microorganism (i.e., a monoculture). The acetogen can be one or more biologically pure strains of acetogenic bacteria, such as one or more naturally occurring strains of acetogenic bacteria, one or more non-naturally occurring strains of acetogenic bacteria, one or more non-naturally occurring strains of acetogenic bacteria produced by genetic modification using acetogenic bacteria as host organism, or one or more non-naturally occurring strains of acetogenic bacteria produced by inserting genes of acetogenic bacteria into a host organism. For example, acetogenic bacteria capable of producing an acid from the gaseous substrate described above include Acetobacterium (Acetoacetate), Moorella (Morera), Clostridium, Pyrococcus, Eubacterium, Desulfobacterium, Cabroxydothermus, Acetogenium, Acetoanaerobium, Butyribaceterium, Peptostreptococcus, Ruminococcus, Oxobacter or Methanosarcina, such as Acetoacetate Corynebacterium sp. No. 446 (FERM P-7017), Acetoacetate Corynebacterium sp. MA-1 (FERM P-8676, P-8676), Acetoacetate Corynebacterium Woody (ATCC 29683, AKA Acetobacterium woodii), Clostridium Asechikamu (DSM 1496), Clostridium Gurikorikamu (ATCC 29797), Clostridium sp. No. 307 (FERM P-7487), Clostridium sp. No. 484 (FERM P-7488), Clostridium sp. No. 68-2 (FERM, P-7367), Clostridium sp. No. 670 (FERM P-8047), Clostridium sp. No. 672 (FERM P-8049), Eubacterium sp. No. 477 (FERM P-8045), Eubacterium-Rimosamu (ATCC 8486, AKA Eubacterium limosum), Eubacterium-Rimosamu (ATCC 10825, AKA Eubacterium limosum), Bacteroides sp. No. 669 (FERM P-8046), Bacteroides sp. No. 671 (FERM P-8048), Bacteroides Ovatasu (ATCC 8483, AKA Bacteroides ovatus), Suporomyusa-Sufaeroidesu (DSM 2875, AKA Sporomusa sphaeroides), Sporomusa ovata (DSM-2662), Acetogenium kivui (ATCC 33488), Morera-Samoasechika (ATCC 31490, AKA Moorella thermoacetica), Morera-Samoasechika (ATCC 35608, AKA Moorella thermoacetica, ATCC 35608), Morera-Samoasechika (ATCC 39073, AKA Moorella thermoacetica), Morera-Samoasechika (ATCC 39289, AKA Moorella thermoacetica), Morera-Samoasechika (ATCC 49707, AKA Moorella thermoacetica), or Morera thermophilus Auto Trophy mosquitoes (ATCC 33924, AKA Moorella thermoautotrophica). Each accession number indicates that the microorganism has been deposited with a depository institution (FERM Patent Organism Depositary Center (FERM); ATCC American Type Culture Collection (ATCC) (www.atcc.org), and DSM German Collection of Microorganisms and Cell Cultures (DSMZ) (www.dsmz.de)). The acetogenic bacteria above can be obtained from the depositories using the accession number provided. In one or more embodiments of the present disclosure, the acetogen includes an A. woodii bacterium.

Inoculating the primary bioreactor with the culture of the acetogenic microorganism can include introducing an aliquot of suspended or separated cells to the primary bioreactor. Inoculating the primary bioreactor can include one or more steps for culturing the inoculum, such as growing an acetogenic microorganism heterotrophically in a nutrient medium to an early stationary phase. In one or more embodiments, inoculating the primary bioreactor includes immobilizing the culture and adding the immobilized culture to the primary bioreactor.

Autotrophic fermentation utilizes a culture medium suitable for efficient conversion of the gaseous substrate to an acid. In one or more embodiments, the culture medium is a liquid or flowable culture medium for use with acetogens, such as homoacetogens. The culture medium can include a complex mixture of peptides and amino acids, nucleotide fractions, organic acids, bicarbonate, minerals, and vitamins in water (e.g., distilled water). Preparing the medium can include purging the medium of oxygen using an oxygen-free or inert gas such as nitrogen or argon (e.g., by sparging).

The source of a portion of the peptides, amino acids, nucleotide fractions, vitamins and minerals for the culture medium can be a yeast extract, beef extract, meat extract, and/or peptone preparation. The composition of the medium can be varied to achieve a specific growth rate, gas consumption rate, acid space-time yield, and acid production rate, according to the guidance described in the Examples below. Additional nutrients can be selected from the group of cofactors for enzymes of the WLP. In one or more embodiments, the culture medium comprises about 2-6 g L⁻¹ yeast extract (e.g., about 4 g L⁻¹ yeast extract), about 1 g L⁻¹ reducing agent, about 5-10 g L⁻¹ bicarbonate, a mineral or trace element, a chelator, a B vitamin or B vitamin precursor, and a buffering agent. The reducing agent can be selected from the group consisting of L-cysteine, thioglycolate, sodium dithionite, dithiothreitol, iron(II) sulfide and sodium sulfide, or a combination thereof. In one or more embodiments of the present disclosure, the medium can comprise a combination of L-cysteine and sodium sulfide as reducing agents. The medium can include a redox potential indicator such as a redox-sensitive dye (e.g., resazurin). The chelator can be selected from the group consisting of ethylenediaminetetraacetic acid and nitrilotriacetic acid, or salts and combinations thereof. The mineral or trace element can be selected from the group consisting of magnesium, calcium, sodium, nitrogen, sulfur, manganese, cobalt, zinc, iron, nickel, aluminum, copper, boron, molybdenum, selenium, and tungsten or salts/chelates or combinations thereof. The B vitamin or B vitamin precursor can be selected from the group consisting of pyridoxine, pantothenate, lipoic acid, nicotinic acid, p-aminobenzoic acid, riboflavin, thiamine, biotin, folic acid, and cobalamin, and salts or combinations thereof. The buffering agent can be selected from phosphates, sulfates, MOPS, TRIS, and TRIZMA, and combinations thereof. The pH of the culture medium can be adjusted to a value within the range of 7-8.2. The culture medium can be provided with an initial carbon source (e.g., fructose or combination of sources) to support heterotrophic growth of the acetogen before the gaseous substrate is introduced into the primary bioreactor.

Anaerobic fermentation of the gaseous substrate produces at least one acid fermentation product. Generally, the composition of the fermentation product(s) will depend on the species of acetogen, or the mixture of acetogens, the composition of the medium (e.g., the concentration of phosphate and presence of carboxylic acids), and the composition of the gaseous substrate. In one or more embodiments of the present disclosure, the culture medium will contain a mixture of fermentation products and metabolites, with acetate forming the major proportion of these products. The mixture may contain additional acids, such as one or more of formic acid, butyric acid, propionic acid, lactic acid, succinic acid, valeric acid, caproic acid, caprylic acid, heptanoic acid, and pyroglutamic acid. The presence and identity of the one or more acids can be confirmed by routine analytical methods (e.g., HPLC). The culture medium can also include fermentation products such as organic compounds other than one or more acids. For example, as a result of CO₂ fermentation by an acetogen, the culture medium can include uracil, acetone, ethanol, and/or the corresponding alcohols of carboxylic acids other than acetate.

Anaerobic fermentation can also include maintaining the pH, temperature, redox potential, and concentration of dissolved gas in the medium based on the growth requirements of the acetogen and acid production. For example, during anaerobic fermentation, the excreted acid can influence the pH of the medium. The pH can be monitored and adapted (e.g., titrating) to maintain the pH of the medium within the desired range during fermentation. Suitable alkaline substances include for adjusting pH can include, e.g., ammonium, alkali metal, or alkali earth metal hydroxides. The bioreactor can be configured to facilitate pH monitoring and adjustment.

The culture medium can be considered to be “ready” for stage II when the acid concentration reaches a target concentration. In one or more embodiments described herein, the target acid concentration can be within the range of about 2.0 to 20 g L⁻¹ culture medium. However, concentrations greater than 20 g L⁻¹ can be suitable as described further below. If PHB is to be prepared in stage II, stage I can be complete when the acetate concentration is about 3.0, 5.0, 7.5, 10, 15, or 20 g L⁻¹ culture medium. Stage I can be terminated by aerating the primary bioreactor, for example. Alternatively, a Bio-GTL method as described herein can be a continuous process, or semi-continuous process, in which only a portion of the acid-containing culture medium is removed for use in stage II upon reaching the target acetate concentration. Fresh culture medium can be introduced to the primary bioreactor to replace the volume removed, or to dilute the acetogen culture, gaseous substrate can be added to maintain pressure at or above 2 bar absolute and stage I acetate production may continue.

### Stage II

### Liquid Substrate

Stage II is initiated when at least a portion of the acid-containing culture medium produced in the primary bioreactor is introduced to a secondary bioreactor as a liquid substrate for microbial conversion to a bioplastic. The step of introducing can include transferring the acid-containing culture medium from the primary bioreactor to the secondary bioreactor by any means. In one or more embodiments described in the present disclosure, the portion is introduced by flowing a portion of the culture medium from the primary bioreactor into the secondary bioreactor (e.g., via a valve, coupler, pipe, tubing, or other conduit configured to receive a liquid from the primary bioreactor and pass the it to the secondary bioreactor). Transferring can include actively pumping to facilitate the delivery of the acid-containing culture medium to the secondary bioreactor. As described above, the acid-containing culture medium can be continuously transferred from the primary bioreactor after a target acid concentration is achieved. Alternatively, the culture medium can be transferred to the secondary bioreactor semi-continuously or in a batch.

In one or more embodiments of the present disclosure, stage II includes adjusting the acid-containing culture medium (e.g., acetate-containing culture medium) to support the growth of a PHA-producing microorganism and the accumulation of PHA in the microorganism. Growth of a PHA-producing microorganism, and the rate at which PHAs are produced, can be influenced by pH. The optimum pH for growth of the microorganism, and highest production rate of PHA can be determined by running the bioreactor, or a separate fermenter (e.g., shaking flasks comprising a culture of the PHA-producing microorganism or single-use bench-top bioreactor), at a range of different pH points. In one or more embodiments described herein, the pH of the culture medium in the secondary bioreactor can be adjusted to be alkaline (i.e., greater than 7). For example, the pH can be adjusted to be slightly alkaline within the range of greater than 7 to less than 8.

The pH of the acid-containing culture medium can be adjusted using pH buffers or alkaline substances known in the art. For example, the culture medium can be adjusted to a slightly alkaline pH using one or more of alkali metal, or alkali earth metal hydroxides, and phosphate buffer. In some instances, the buffer or alkaline substance is selected from phosphate- and/or nitrogen-free compounds to facilitate nutrient-limited growth conditions that induce microbial PHA production and accumulation.

In one or more embodiments of the present disclosure, adjusting the culture medium for PHA production includes adjusting the acid concentration and pH. For example, the optimum acetate concentration for growth of the microorganism, and highest production rate of PHB can be determined by running the bioreactor, or a separate fermenter comprising a portion of the culture medium of the first stage (e.g., shaking flasks comprising a culture of the PHA-producing microorganism), at a range of different acetate concentrations and different pH. In one or more embodiments described in the present disclosure, when the desired PHA is PHB, the acetate concentration of the culture medium in the secondary bioreactor can be adjusted to be less than about 20 g L⁻¹ acetate (e.g., by dilution with fresh culture medium suitable for use in stage I) and the pH can be adjusted to be slightly alkaline, as described above. For example, the acetate concentration of the culture medium in the secondary bioreactor can be adjusted to be within the range of about 2.0 to 20 g acetate L⁻¹, including about 5.0, 7.5, 10, 15 or 20 g acetate L⁻¹ for microbial growth and PHB production.

In one or more of the embodiments described herein, the composition of the culture medium of stage I is based on, not only its suitability for anaerobic fermentation, but also on its suitability for microbial growth in stage II of a Bio-GTL process. For example, a candidate culture medium can be initially prepared for optimal production of acetate by a specific acetogenic microorganism, aliquots of the candidate culture medium can be supplemented with acetate, or the acid needed to produce the desired bioplastic, at different concentrations, each aliquot can be inoculated with a microorganism capable of accumulating PHA using the acid as a substrate, and the cell density and PHA accumulation in each aliquot can be measured at different time points. The acid concentration, nutrient concentration, and pH of the candidate culture medium can be methodically adjusted to formulate a culture medium that is suitable for achieving a PHA cell content greater than 24% by weight within about 10 to 18 hours of growth. In one or more embodiments, the aliquots can be inoculated with the PHA-producing microorganism before the acetate is added. In one or more embodiments, the candidate culture medium can achieve PHA cell content greater than 24% by weight after about 10 hours of growth without requiring the addition of a growth-limiting nutrient to the aliquots (i.e., the acid-containing culture medium is nutritionally complete). A pH adjustment may be the only adjustment needed for optimal PHA accumulation. Providing a single medium composition that requires only an adjustment of pH enables a continuous setup with stages I and II being connected to each other in a looped system (e.g., a closed-loop) and cost-saving recycling of culture medium.

In one or more embodiments of the present disclosure, adjusting the culture medium to support PHA-production includes aerating or oxygenating the culture medium. For example, a stream of gaseous oxygen, free of nitrogen, can be introduced into the secondary bioreactor to promote PHA production. The stream of gaseous oxygen can be supplied from an oxygen source that has been obtained from a renewable resource, such as oxygen produced by electrolysis with photovoltaic energy. The renewable resource for O₂ can be the same resource used to generate H₂ for stage I, as described above. Oxygenating or aerating can include agitating the culture medium. The level of dissolved oxygen in the culture medium can influence microbial growth and metabolite production in a bioreactor. Agitation improves mass and oxygen transfer and maintains homogeneous chemical and physical conditions. The optimal agitation and oxygenation or aeration can be determined on a bench-scale, and then scaled up based on volumetric oxygen transfer coefficient, volumetric power consumption, agitation speed (e.g., impeller tip speed), and mixing time.

### Aerobic Fermentation

Stage II utilizes a culture of a PHA-producing microorganism to convert the acid to PHA by aerobic fermentation. The PHA-producing microorganism is different from the anaerobic microorganism. The culture can be a mixed culture of two or more PHA-producing microorganisms or a culture comprising a single PHA-producing microorganism (i.e., a monoculture). The PHA-producing microorganism can be one or more biologically pure strains of PHA-producing bacteria, such as one or more naturally occurring strains of PHA-producing bacteria, one or more non-naturally occurring strains of PHA-producing bacteria, one or more non-naturally occurring strains of PHA-producing bacteria produced by genetic modification of a host organism, or one or more non-naturally occurring strains of PHA-producing bacteria produced by inserting genes of PHA-producing bacteria into a host organism. In one or more embodiments of the present disclosure, the PHA-producing microorganism is selected from the group consisting of Cupriavidus necator (ATCC 17697), Cupriavidus necator H16 (ATCC 17699), Azohydromonas lata (ATCC 29712) Pseudomonas putida mt-2 (ATCC 33015), Bacillus spp. type strains, Corynebacterium glutamicum (ATCC 15990), Corynebacterium hydrocarboxydans (ATCC 21767), Nocardia lucida (NCIMB 10980), Rhodococcus sp., and transgenic microorganisms expression genes capable of converting the acid to at least one PHA (e.g., microorganisms engineered to express poly-β-hydroxybutyrate polymerase, or another enzyme involved in PHA biosynthesis). In one or more embodiments, the PHA-producing microorganism is C. necator H16, i.e., the Gram-negative lithoautotrophic bacterium belonging to the β-subclass of the Proteobacteria, also known as Ralstonia eutropha H16, Alcaligenes eutrophus, “Hydrogenomonas eutropha”, Ralstonia eutropha, and Wautersia eutropha.

The secondary bioreactor is inoculated with a culture of a PHA-producing microorganism after the acid-containing culture medium has been adjusted as described above. Inoculating can be accomplished by adding cells that have been separated from an initial growth medium. The cells can be washed (e.g., with a phosphate buffer) before addition. The cells can be free of phosphate or nitrogen-containing solutions, or the PHA-producing microorganism can be added to the secondary bioreactor as a suspension of cells in buffer or fresh culture medium (e.g., the fresh culture medium described above for stage I). The secondary bioreactor can be inoculated to achieve a target cell density (OD₆₀₀) of at least 0.1, such as about 1-2, or about 1.5, 1.6, 1.7, or 1.8).

In one or more embodiments, inoculating the secondary bioreactor includes culturing the inoculum by growing a PHA-producing microorganism in a nutrient medium that has a different composition than the culture medium utilized for stage I. The PHA-producing microorganism can be pre-cultured from frozen stock in a rich medium to form a first seed culture. This culture can be transferred into a second seed culture comprising complete growth medium supplemented with a carbon source (e.g., gluconate, monosaccharides, and disaccharides, such as fructose, sucrose, and glucose; polyols such as glycerol; fats; and oils). Inoculating the secondary bioreactor can include preparing the inoculant from the growth medium by harvesting and washing the PHA-producing microorganism of the second seed culture to remove the complete medium and facilitate nutrient limited conditions that promote PHA production and accumulation.

Aerobic fermentation can also include maintaining the pH, temperature, and concentration of dissolved oxygen in the medium based on the growth requirements of the PHA-producing microorganism and the rate of PHA production. For example, during fermentation, the consumption of the acid can result in a change in the pH of the medium. Alternatively, in a continuous or semi-continuous process, the pH of the medium in the secondary bioreactor can be altered by the continuous or semi-continuous addition of acid-containing medium from the primary bioreactor, and/or continuous or semi-continuous harvesting of cells containing the target PHA content (e.g., % by weight). Thus, the method can include monitoring and adapting the pH (e.g., titrating) to maintain the pH of the medium within the desired range during batch, semi-continuous or continuous fermentation, and the bioreactor can be configured or adapted to facilitate maintaining pH (e.g., the secondary bioreactor can include a pH control system). The dissolved oxygen content, temperature, and nutrient homogeneity can be controlled, in part, by agitating the culture medium in the secondary bioreactor within a range of rotation speeds, e.g., from about 100 to about 3,000 rpm or about 150 to about 2,800 rpm. The secondary bioreactor can be agitated by shaking (e.g., flask) or stirring (e.g., stirred reactor).

During stage II, the culture of the PHA-producing microorganism converts the acid to a PHA. PHAs are storage compounds that are accumulated by a diverse range of microorganisms under unbalanced growth conditions (e.g., excess of a carbon source with simultaneous depletion of an essential growth element). The PHA is accumulated in inclusion bodies in the PHA-producing cell. The monomer composition of microbially synthesized PHAs can vary based on the composition of the acid-containing culture medium and the microbial strain used. In one or more embodiments of the present disclosure, if the culture medium supports the production of a mixture of PHAs, the mixture includes a major proportion of polyhydroxybutyrate (PHB), such as poly(3-hydroxybutyrate) (PHB). In some cases, only PHB is produced. In addition to PHA mixtures, the PHA-producing microorganism can convert the carbon sources into PHA copolymers (e.g., poly(3-hydroxybutyrate-co-3-hydroxy valerate) and poly(3-hydroxybutyrate-co-4-hydroxybutyrate)). The side chain of the PHA can be saturated or not, and can possess branched, aromatic, halogenated, and epoxidized monomers. The side chain-length of the PHA can also vary based on the type of PHA-producing microorganism. For example, small chain PHAs are characterized by of 3-5 carbon atoms and are synthesized by a wide range of bacteria such as C. necator, and medium chain PHAs are composed of monomers having 6-14 carbon atoms and are synthesized by Pseudomonas species.

Stage II fermentation can proceed until the culture reaches a target end-point. For example, aerobic fermentation can be considered “complete” or the PHA “ready for harvest” when the culture is in the stationary phase of the growth curve. In other embodiments, stage II continues for a fixed duration (e.g., about 8-18 hours after inoculation, such as about 10, 12, 14, 16 or 18 hours after inoculation). Alternatively, aerobically fermenting can include growing the PHA-producing microorganisms until the culture achieves a target cell density (e.g., OD₆₀₀ of about 1-12, such as about 4-12, 6-12, or 8-12) or until a target PHA content in the cells has been achieved. PHA content can be determined in samples removed from the secondary bioreactor during fermentation. After removal, intracellular PHA can be stained using a fluorescent dye, such as Nile red, for fluorescent microscopy analysis (e.g., using an excitation λ=562/40 nm, and detecting the emission of λ=594 nm with a long pass filter). The cell content of PHA by weight can be calculated relative to the cell dry weight of the sample. In some cases, aerobic fermentation continues until the PHA-content is greater than about 24%, such as greater than about 30%, 35%, 40%, 45%, or 50% and up to about 80% of the cell dry weight.

Upon reaching the desired endpoint of aerobic fermentation, stage II can include harvesting the PHA-containing biomass and extracting the PHA. For example, the process can include separating the biomass from the culture medium by filtering or centrifugation. In one or more embodiments of the present disclosure, the separated culture medium can be introduced into the primary bioreactor to be utilized in another cycle of anaerobic fermentation, i.e., recycled. The separated culture medium can be autoclaved, de-oxygenated (e.g., sparged), and/or supplemented with nutrients that may have been consumed during one or more stages of fermentation before introduction to the primary bioreactor. Extraction of PHA from the PHA-containing biomass can be accomplished using known methods. For example, PHA-containing cells can be washed, freeze-dried, and subjected to acidic methanolysis. Alternatively, washed PHA-containing cells can be lyophilized, micronized, and treated with an aqueous hypochlorite solution to precipitate PHA polymers. After extraction, the isolated polymers can be quantitatively and/or qualitatively analyzed. In some cases, the content, composition, and/or molecular weight of PHA homopolymers and copolymers produced by the PHA-producing microorganism can be analyzed by gas chromatography or gel permeation chromatography using known standards (e.g., poly(3-hydroxybutyrate) of a known molecular weight).

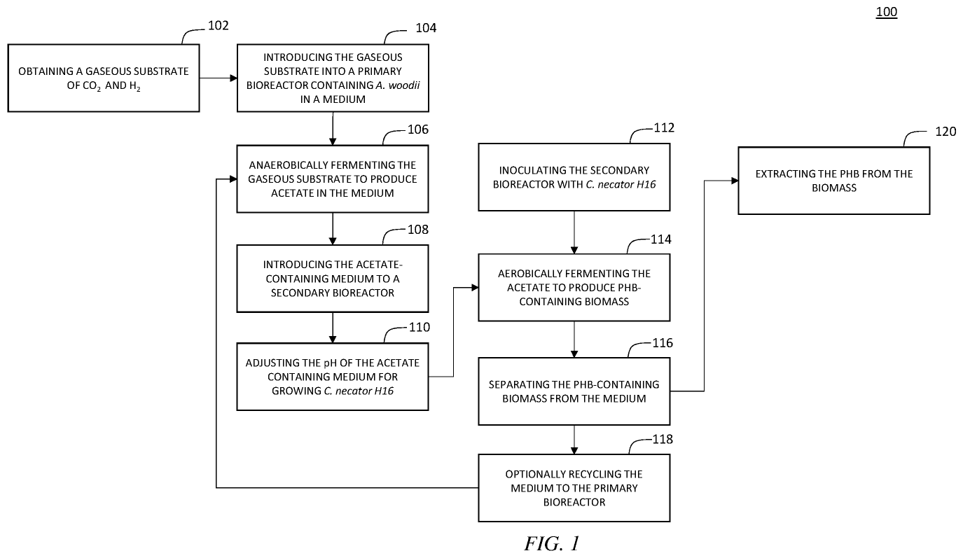

FIG. 1 provides a flowchart of method 100 for capturing and converting CO₂ into PHB, as an exemplary bioplastic, according to one or more embodiments of the present disclosure. Within block 102, method 100 includes obtaining a gaseous substrate including CO₂ and H₂. The gaseous substrate can be CO₂ and H₂ alone, or in admixture other gases (e.g., syngas, carbon monoxide, methane, and other gases present in industrial emissions, as described above). The gaseous substrate can be obtained by providing one or more gas tanks (e.g., a mixed-gas tank). In a mixed-gas tank the ratio of H₂:CO₂ can be about 75:25 to about 95:5, such as about 85:15. Block 102 can also include blending one or more gas sources. For example, each gas source can be configured to flow into a mixing chamber (e.g., a small vessel or section of pipe, provided with a mixing device to promote homogenization of each gas to form a mixed-gas substrate with H₂ and CO₂ at a ratio described above.

In an optional embodiment of block 102, the gaseous substrate comprising CO₂ and/or H₂ gas is obtained from one or more renewable resources. For example, as described above, H₂ can be obtained via photovoltaic electrolysis. The gaseous substrate from the renewable resource may be filtered, scrubbed, or otherwise treated using known methods in preparation for anaerobic fermentation.

The gaseous substrate is introduced to a primary bioreactor, as shown in block 104. The primary bioreactor can be any suitable bioreactor for anaerobic fermentation. The primary bioreactor can be a sealable high-pressure reactor configured to receive a stream gaseous substrate and achieve a target pressure. Block 104 can include steps of sealing and pressurizing the reactor with to a pressure greater than or equal to 2 bar (absolute), such as 2-5.5 bar absolute, and maintaining the pressure during anaerobic fermentation. In one or more embodiments, block 104 can include maintaining the pressure in the primary bioreactor as subsequent steps of method 100 are performed.

The primary bioreactor is configured for anaerobic fermentation using a culture of an acetogen in a medium (e.g., a culture medium as described above). Although block 104 describes a bioreactor comprising a culture of A. woodii, this embodiment is not limiting. The acetogen can be selected from the strains of acetogenic microorganisms described above. Block 104 optionally includes a step of inoculating the primary bioreactor with the acetogen (e.g., with an inoculum grown heterotrophically in a nutrient medium to an early stationary phase). Block 104 can also include one or more steps for preparing the medium for use in the primary bioreactor (e.g., step-wise mixing, sparging to remove oxygen, and autoclaving).

As shown in block 106, method 100 includes anaerobically fermenting the gaseous substrate to produce acetate in the medium. However, additional acids and other fermentation products can be produced, even in major amounts. Anaerobically fermenting can be performed as a batch, semi-continuous, or continuous process, which can include monitoring the formation of metabolic products. In some cases, monitoring includes identifying and/or quantifying metabolic products using analytic tools, such as HPLC and GC-MS.

Within block 106, anaerobically fermenting can include monitoring and maintaining the pressure of the gaseous substrate within the primary bioreactor. When the gaseous substrate is consumed, the gas pressure decreases. Maintaining the pressure can include building the pressure back to the set point (e.g., ≥2 bar absolute) by flowing an amount of gaseous substrate equal to the amount consumed from the gas source into the primary bioreactor. For example, maintaining the pressure can include delivering the gaseous substrate using pressure regulation to build the pressure back to the initial pressure.

Anaerobically fermenting can include maintaining the pH, temperature, redox potential, and concentration of dissolved gas in the medium based on the growth requirements A. woodii and acetate production. For example, during anaerobic fermentation acetate production results in a change in the pH of the medium. Thus, block 106 can include monitoring and adapting the pH to maintain the pH of the medium within the desired range during fermentation.

Anaerobically fermenting can also include agitating the medium. In one or more embodiments, method 100 includes shaking or stirring the primary bioreactor within a range of rotations speeds, e.g., from about 100 to about 1200 rpm or about 150 to about 500 rpm.

To convert the acetate (or, alternatively, a different acid or mixture of acids) to biodegradable bioplastic, method 100 includes introducing the acid-containing medium from the primary bioreactor into a secondary bioreactor, as shown in block 108. For example, the step of introducing the medium into the secondary bioreactor can be performed when the medium comprises at least about 2.5 g L⁻¹ acetate. In one or more embodiments of the present disclosure, the concentration of acetate is less than about 20, 15, 10, 7.5, 5 or 3 g L⁻¹, and at least about 2.5 g L⁻¹ (e.g., within the range of 2.5-7.5 g L⁻¹) when at least a portion of the medium is removed from the primary bioreactor and introduced into the secondary bioreactor.

When the acetate-containing medium has been introduced to the secondary bioreactor, method 100 includes adjusting the pH of the acetate-containing medium for the growth of C. necator H16, as shown in block 110. While C. necator H16 is described in this block, this species of PHA-producing microorganism is merely exemplary. The pH can be adjusted to support the growth of any of the PHA-producing microorganisms described above. In one or more embodiments, adjusting the culture medium can include adding a buffer, acid or alkaline substance to the medium. For example, the pH of the culture medium can be adjusted with an alkaline substance selected from phosphate- and/or nitrogen-free compounds, as described above.

When the pH of the acid-containing medium in the secondary bioreactor is adjusted, method 100 includes inoculating the secondary bioreactor with C. necator H16 (or the PHA-producing microorganism for which the pH was adjusted in block 110) as shown in block 112. The secondary bioreactor can be inoculated to achieve a target optical density (OD₆₀₀). Block 112 can include one or more steps for pre-culturing and transferring the inoculant as described above (e.g., growing one or more seed cultures, washing, etc.).

Method 100 includes aerobically fermenting the acetate present in the medium to produce PHB. Although block 114 describes PHB production using acetate as the substrate, other PHAs can be produced, depending on the PHA-producing organism and the components of the medium. Aerobically fermenting can be performed as a batch, semi-continuous, or continuous process, and can include monitoring pH, acetate uptake, optical density or PHB accumulation in C. necator H16 cells. Aerobically fermenting can include adjusting pH or physical conditions (e.g., temperature, agitation, and aeration) to enhance growth or minimize growth inhibition. For example, the method can include agitating the culture medium in the secondary bioreactor within a range of rotation speeds, e.g., from about 100 to about 3,000 rpm or about 150 to about 2,800 rpm. Aerobically fermenting the acid proceeds until one or more of the endpoints described above is reached (e.g., the target concentration of active biomass (OD₆₀₀), stationary growth phase, elapsed time, or PHB-content (% cell dry weight)).

After the end-point for aerobic fermentation is achieved, method 100 can optionally include separating the PHB-containing biomass from the from the medium, as shown in block 116. Separating the biomass from the medium can be performed using known methods, such as filtration and/or centrifugation (e.g., continuous flow centrifugation).

Method 100 can optionally include recycling the medium separated in block 116 to the primary bioreactor as shown in block 118. The primary bioreactor can comprise a continuous culture of A. woodii for anaerobically fermenting the gaseous substrate. Alternatively, the primary bioreactor can be clean and ready for inoculation with a culture of A. woodii before or after the separated medium is introduced to the primary reactor. Recycling the culture medium can include supplementing the separated medium with nutrients that may have been depleted during fermentation of the acid. Recycling the medium can also include additional filtering steps, steps for purging the medium of oxygen, and/or determining the redox potential of the medium prior to introducing the separated medium into the primary bioreactor.

Method 100 can optionally include extracting PHB from the PHB-containing biomass, as shown at block 120. Methods of extracting PHAs such as PHB are described above and in the examples below.

The following Examples are intended to illustrate the above invention and should not be construed as to narrow its scope. One skilled in the art will readily recognize that the Examiners suggest many other ways in which the invention could be practiced. It should be understood that numerous variations and modifications may be made while remaining within the scope of the invention.

### Example 1

### A Two-Stage Biological Gas to Liquid Transfer Process (Bio-GTL) to Convert CO₂ into Bioplastic

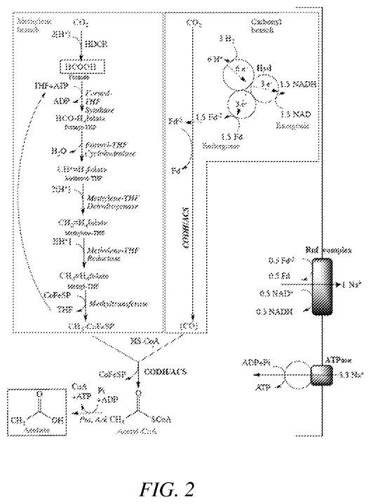

Microbial CO₂ fermentation can be performed by acetogens, a class of anaerobic microorganisms that synthesize acetyl-CoA and cell carbon from CO₂ using the Wood-Ljungdahl pathway (WLP) to produce acetic acid. As shown in FIG. 2, during autotrophic acetogenesis, acetate is formed from four H2 and two CO₂, using the methylene branch in combination with the carbonyl branch of the pathway, according to the following equation: 2CO₂+4 H₂→CH₃COOH+H₂O+0.3 ATP (Wood 1991). The Rnf complex pumps one Na⁺ ion outside the cell per electron, which is gained from the oxidation of reduced ferredoxin (Fd²⁻). The resulting ion gradient is consumed by the ATPase resulting in which is gained from the oxidation of Fd²⁻. The resulting ion gradient is consumed by the ATPase resulting in the formation of 0.3 ATP per Na⁺ ion.

The two-stage biological process that transfers gas to liquid (Bio-GTL) is a promising technology that combines two microbial processes: (i) the metabolism of gaseous substrates (e.g., CO₂), and (ii) the synthesis of bioproducts. Bio-GTL is suitable to produce acetic acid from CO₂ fermentation and upgrade it into a more valuable product. Thus, Bio-GLT bypasses the expensive recovery process of acetate in the aqueous phase, reducing the production costs.

In the Bio-GTL two-stage process described in these Examples, CO₂ is first converted into acetate by an autotrophic acetogen using H2:CO₂ (85:15) gas mixture. In an example, the strictly anaerobic model strain Acetobacterium woodii utilizes the Wood-Ljungdahl pathway for acetate formation. Elevated pressure (≥2.0 bar) in a high-pressure stirrer tank is used to increase H₂-solubility in water. This is highly beneficial, since the aqueous H₂ concentration was found to be rate limiting for this process.

In a second stage, acetate is converted into a higher value compound (bioplastic) under aerobic conditions. In the following examples, the metabolically highly versatile strain Ralstonia eutropha H16 (AKA Cupriavidus necator H16) serves as a model strain for the production of PHB from acetic acid as the only carbon source. The aerobic conditions allow the bacteria to generate energy from acetate oxidation, and thus, permits the formation of endothermic products. The two-stage process facilitates the combination of highly efficient acetate formation from CO₂/H₂ mixtures with anaerobic strains and the aerobic conversion of acetate into viable products, and thereby permitting efficient conversion of CO₂ into products of value.

The kinetics (microbial uptake and conversion) and thermodynamics (efficiencies) of both stages were evaluated under several bioprocessing conditions to address the influence of several parameters on efficiencies and time-space yields, e.g., the partial pressures of H₂ and CO₂, steady-state acetate levels and pH levels in the individual stirred tank batch reactors. The results show a single culture medium can be used in both stages without any initial adjustment of nutrients.

The following examples present a Bio-GTL microbial process to convert CO₂ into polyhydroxybutyrate (PHB) using H2 as an electron donor, using two metabolically different strains. Stage one uses the acetogen A. woodii to convert CO₂ into acetic acid. In stage two, R. eutropha H16 converts acetate into PHB. Compared to the method by Lagoa-Costa et al. (full citation above), the approach described in the present disclosure has at least two advantages: (i) an increase in gas-to-liquid mass transfer by applying high-pressure conditions without excessive gas consumption and (ii) reduced experimental time.

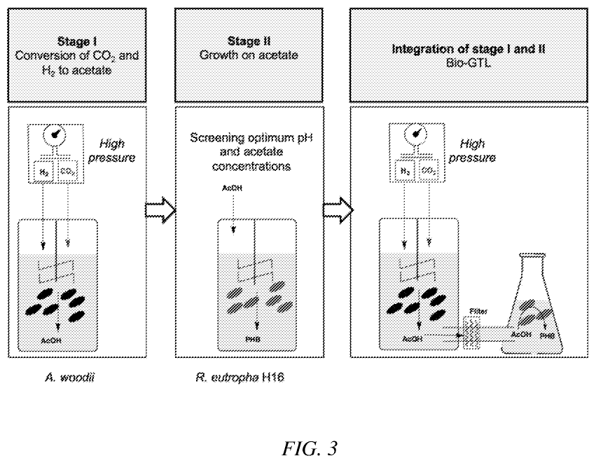

To determine the effect of pressurized substrate on the gas-to-liquid mass transfer for CO₂ capture during stage one, A. woodii was grown in a high-pressure stirred-tank reactor pressure at 2.0 and 5.5 bar absolute. For the second stage, maximum growth and acetate uptake were evaluated by growing R. eutropha H16 in parallel bioreactor fermentations on a milliliter-scale setup under a wide range of acetate concentrations and pH values. The kinetics (acetate production rates and conversion rates of acetate into PHB) and thermodynamics (energy efficiencies), as well as the conditions with highest energy efficiency were integrated into a Bio-GTL using one medium for both microorganisms (FIG. 3). These results support sustainable bioplastics production from CO₂.

### Materials and Methods

Microorganisms

A. woodii (DSM 1030) and R. eutropha H16 (DSM 428, AKA C. necator H16) strains were purchased from the DSMZ (Braunschweig, Germany). Fresh cultures were prepared from aliquots and stored at −80° C.

Media and Culturing

Anaerobic pre-cultures of A. woodii were grown heterotrophically to an early stationary phase at 30° C., 160 rpm in shaking flasks (Incu-Shaker Mini™, Benchmark, N.J., USA) placed in a N₂ glovebox (InerTec AG, Grenchen, Switzerland) in medium A1, consisting of the components listed in Table 1.

TABLE 1
‘A1’ medium.

| | CONCENTRATION | |
|---|---|---|
| MEDIUM COMPONENT | (L⁻¹) | |
| NH₄Cl | 1.0 | g |
| KH₂PO₄ | 0.33 | g |
| K₂HPO₄ | 0.45 | g |
| MgSO₄•7H₂O | 0.16 | g |
| yeast extract (Sigma-Aldrich, Missouri, USA); | 2.0 | g |
| NaHCO₃ | 10 | g |
| cysteine-HCl•H₂O | 0.5 | g |
| Na₂S•9H₂O | 0.5 | g |
| nitrilotriacetic acid | 30 | mg |
| MnSO₄•H₂O | 10 | mg |
| NaCl | 20 | mg |
| FeSO₄•7H₂O | 2.0 | mg |
| CoSO₄•7H₂O | 3.6 | mg |
| CaCl₂•2H₂O | 2.0 | mg |
| ZnSO₄•7H₂O | 3.6 | mg |
| CuSO₄•5H₂O | 0.2 | mg |
| KAI(SO₄)2•12H₂O | 0.4 | mg |
| H₃BO₃ | 0.2 | mg |
| Na₂MoO₄•2H₂O | 0.2 | mg |
| NiCl₂•6H₂O | 0.5 | mg |
| Na₂SeO₃•5H₂O | 6 × 10⁻³ | mg |
| Biotin | 4 × 10⁻⁵ | mg |
| folic acid | 4 × 10⁻⁵ | mg |
| pyridoxine-HCl | 0.2 | mg |
| thiamine-HCl•2H₂O | 0.1 | mg |
| riboflavin | 0.1 | mg |
| nicotinic acid | 0.1 | mg |
| D-Ca-pantothenate | 0.1 | mg |
| Cyanocobalamine | 2 × 10⁻⁶ | mg |
| p-aminobenzoic acid | 0.1 | mg |
| lipoic acid | 0.1 | mg |
| fructose | 10 | g |

The initial pH was 7.0, and the inoculum was 1 mL of frozen cells (cryo stock, prepared by 0.9 mL early stationary phase cultures reserved in 0.1 mL DMSO) in 200 mL medium ‘A1’.

Batch processes for autotrophic fermentation of A. woodii were performed in a high-pressure reactor using 1 L medium ‘A2’ (initial pH 7.0) in which the concentrations of yeast extract, vitamins and trace elements used to prepare ‘A1’ (Table 1) were doubled to avoid growth limitation and the NaHCO₃ concentration was reduced from 10 g L⁻¹ to 5 g L⁻¹ as mentioned in the literature. Later, medium ‘A2’ was modified to 1 L medium ‘A3’ by increasing the yeast extract concentration 50% (from 4 g L⁻¹ to 6 g L⁻¹), which was used for autotrophic fermentation with an initial pH of 7.0. The inoculum for medium ‘A2’ and ‘A3’ was harvested from 200 mL pre-cultures in early stationary phase by centrifugation (Eppendorf centrifuge 5430 R, Eppendorf, Hamburg, Germany; 4,500×g, 10 min, 4° C.). The pellet was dissolved in 10 mL of medium ‘A2’. The inoculum was anaerobically transferred with a syringe into the reactor. To increase the gas-to-liquid mass transfer for CO₂ capture, 2.0 bar and 5.5 bar absolute pressure were applied on medium ‘A2’. Medium ‘A3’ was applied under 5.5 bar only. The metabolic products were identified, quantified (by HPLC and GC-MS, as described later) and the energy efficiency of H₂ conversion to acetic acid was calculated.

Pre-cultures of R. eutropha H16 were grown overnight (the inoculum was a loop-full from cryo stock) at 30° C., 140 rpm (Genesys™ 20, Thermo Spectronic™, Neuss, Germany) in 1 L rich medium (peptone 5 g L⁻¹, meat extract 3 g L⁻¹). 10% (v/v) of the overnight culture were transferred in 1 L (end volume) minimal medium ‘R1’ (initial pH 7.0) consisting of (NH₄)₂SO₄ 3 g L⁻¹; KH₂PO₄ 1.5 g L⁻¹; Na₂HPO₄ 4.45 g L⁻¹; MgSO₄ 0.097 g L⁻¹; CaCl₂6H₂O 0.02 g L⁻¹; FeSO₄.7H₂O 0.02 g L⁻¹; MnCl₂.4H₂O 24 μg L⁻¹; ZnSO₄.7H₂O 528 μg L⁻¹; Na₂MoO₄.2H₂O 150 μg L⁻¹; CuSO₄.5H₂O 240 μg L⁻¹; COCl₂.6H₂O 90 μg L⁻¹; H₃BO₃ 864 μg L⁻¹; NiCl₂ 24 μg L⁻¹; 30 g L⁻¹ fructose as carbon source. The cultures were grown for 15 h at 30° C., 140 rpm (Genesys™ 20). The influence of different acetic acid concentrations on cell growth was screened in single-use stirred-tank bioreactors on a milliliter scale (bioREACTOR 48, 2 mag AG, Munich, Germany). Inoculum from ‘R1’ medium was inoculated to a final OD₆₀₀ 1.6-1.8 in 12 mL ‘R2’ medium consisting of (NH₄)₂SO₄ 1.83 g L⁻¹; KH₂PO₄ 1.47 g L⁻¹; K₂HPO₄ 2.46 g L⁻¹; MgSO₄.7H₂O 0.27 g L⁻¹; CaCl₂.2H₂O 0.03 g L⁻¹; FeSO₄.7H₂O 0.02 g L⁻¹; MnCl₂.4H₂O 24 μg L⁻¹; ZnSO₄.7H₂O 528 μg L⁻¹; Na₂MoO₄.2H₂O 150 μg L⁻¹; CuSO₄.5H₂O 240 μg L⁻¹; COCl₂.6H₂O 90 μg L⁻¹; H₃BO₃ 864 μg L⁻¹; NiCl₂ 24 μg L⁻¹. The batch fermentation was carried out with 30 g L⁻¹ fructose as a starting carbon source for 8 h at 30° C. The pH was varied in the following range: pH 6.5, pH 7.0, pH 7.5, pH 8.0. pH control was performed using 12% (w/w) NH₄OH and 0.5 N H3PO₄. After 8 h, different concentrations of acetic acid were supplied in the form of sodium acetate (pKa 4.75) to reach the following final concentrations: 2.5 g L⁻¹, 5.0 g L⁻¹, 7.5 g L⁻¹, 10 g L⁻¹, 15 g L⁻¹, 20 g L⁻¹ and incubated for an additional 10 h. The pH control was performed using 0.5M KOH and 0.5M H3PO₄ under nitrogen-limited conditions. The optical density (OD₆₀₀) was measured at the end of each run (Genesys™ 20).

The integration study with R. eutropha H16 was performed in 50 mL medium obtained from A. woodii after high-pressure fermentation. Prior to integration, A. woodii was filtered from the medium. R. eutropha H16 was incubated in shaking flasks at 30° C., 140 rpm (initial pH 7.5). The R. eutropha H16 inoculum was pre-grown in medium ‘R1’ over-night. Prior to integration, to remove media ‘R1’ residues, the cells were centrifuged (Eppendorf centrifuge 5430 R, 4,500×g, 10 min, 4° C.) and washed once with phosphate buffer (30 mM, pH 7.5). The amount of inoculum was chosen to reach a final OD₆₀₀ of 1.5 in the experimental vessel.

High-Pressure Reactor

Autotrophic fermentation of A. woodii was performed in a high-pressure stirred-tank “ecoclave 075” type 1B/1.6 L reactor without baffles (BÜCHIGLASUSTER®, Uster, Switzerland). Dimensions and system operating conditions were 60×45×100 cm width/depth/height; −1/+6 bar min./max. pressure; −20/+200° C. min./max. temperature controlled with a stainless-steel 2mag PT100 external temperature sensor (2mag-USA, Florida, USA). A mixed-gas tank of H₂:CO₂ (85:15) was connected to the system and controlled by a pressure regulator (bpc 2, Büchi pressflow controller; BÜCHIGLASUSTER®). The pH control system consisted of dosing pumps (ProMinent® The Delta®, Pennsylvania, USA) for maximum backpressure of 25 bar (pumps controlled by frequency) and an analog pH glass sensor (Orbisint CPS 11; Endress+Hauser, Switzerland). Since the pH decreased due to acetic acid production, 0.1 M KOH was used to adapt the pH. To eliminate any traces of oxygen in the medium, the sterile medium was stripped with argon for 10 purges, followed by 10 purges of the gas mixture (0.5 mg L⁻¹ resazurin (7-Hydroxy-3H-phenoxazin-3-one 10-oxide) was used as indication of oxygen absence).

The reactor vessel was autoclaved and afterwards filled with 1 L filtered medium via a peristaltic pump (Thermo Scientific Masterflex® PS, Illinois, USA). The agitation speed was set to rpm using a stirrer shaft Dm10×294 with turbine Dm45 (BÜCHIGLASUSTER®), and the temperature was controlled to 30° C. The fermentation was performed in batch mode, where the gas from the mixed-gas tank was fed in the reactor to a set pressure using a volumetric gas dosing system (bpc 2) (BÜCHIGLASUSTER®). When the pressure of the gas dropped due to the uptake by the microorganisms, the drop was recorded in liters (L) by the volumetric gas dosing system (bpc 6002, BÜCHIGLASUSTER®) and the same value was fed to the reactor from the tank to build the pressure again.

Parallel Fermentations in Milliliter-Scale Stirred-Tank Bioreactors

Parallel fermentation experiments were performed on a milliliter-scale in sterile single-use stirred-tank bioreactors with an initial volume of 10 mL at 30° C. (bioREACTOR, 2mag AG, Munich, Germany). Dissolved oxygen (DO) and pH were monitored by fluorimetric sensors immobilized at the bottom of each single-use bioreactor using fluorimetric readers (MCR 8*2 v5, PreSens GmbH, Regensburg, Germany). The pH of each bioreactor was adjusted with 12% (w/w) NH₄OH and 0.5 M H₃PO₄ during the first 8 h, then with 0.5 M NaOH (nitrogen limitation conditions to promote PHB production) and 0.5 M H₃PO₄ by a liquid handling system (Freedom EVO®, Tecan GmbH, Crailsheim, Germany) controlled by the software fedbatchXP (DASGIP®—an Eppendorf company, Jülich, Germany). The minimal volume of base addition was set to 10 μL (0.1% of the reaction volume). Substrate feeding was done manually. Samples were withdrawn automatically with the liquid handling system at preset process times and were used for the determination of bacterial growth and of concentrations of acetic acid.

The oxygen transfer rate was kept sufficiently high by using gas-inducing stirrers operated at an agitation speed of 2,800 rpm and a headspace aeration of 0.1 L sterile air min⁻¹ reactor⁻¹. Liquid volume loss by evaporation was avoided by using sterile air that was saturated with water at room temperature (25° C.), and the headspace cooling was adjusted to 20° C.

Fermentation Product Identification and Quantification

High-performance liquid chromatography (HPLC, Agilent 1200 series, California, USA) was used to quantify the fermentation products. The HPLC was equipped with ICE-Coregel 87 H3 column (Transgenomic®, Minnesota, USA), and the eluent was 0.008 M H₂SO₄ solution at a flow rate of 0.8 mL min⁻¹. A UV-VIS detector was used at λ-214 nm and 35° C. The acetic acid and formic acid were spiked on the HPLC to identify their retention times. The unknown metabolic products were identified by detecting their retention times and collecting the fractions using an HPLC equipped with a fraction collector (HPLC, Agilent 1200 series, G1364B Fraction Collector, California, USA). The fractions were freeze-dried (VirTis® BenchTop™ 6K, New York, USA), treated with methoxyamine and then derivatized using trimethysilanol (TMS) reagent (BSTFA, Thermo Fisher Scientific™, Massachusetts, USA). The fractions were analyzed using gas chromatography coupled with mass spectrometry (GC/MS 7890/5975C; Agilent Technologies, California, USA) equipped with a DB-5 capillary column (30 m, 0.25 mm ID and 0.25 μm film thickness, Agilent Technologies). The injection volume was 1 μL. The column ran with an initial temperature of 50° C. for 1 min, a ramp rate of 10° C. min⁻¹ up to 290° C. for 35 min (total run time 60 min). The flow rate was 1.5 mL min⁻¹. The GC/MS raw data file of each sample was de-convoluted using the AMDIS software (chemdata.nist.gov/mass-spc/amdis/downloads/) and each compound detected was identified by the NIST 11 library database (Agilent Technologies).

PHB Quantification with GC-FID

Cultures (2 mL) were centrifuged (Eppendorf centrifuge 5430 R, 6,500×g, 50 min, 4° C.) and then washed with double distilled water (ddH₂O) to decrease residual salts from the medium. Subsequently, the cells were re-suspended in water, shock-frozen in liquid N₂ and then lyophilized. Afterwards, methanolysis was performed by dissolving the dried cells in 2 mL of 6% (v/v) sulfuric acid in methanol solution containing 100 mg L⁻¹ of sodium benzoate as an internal standard. Then 2 mL of chloroform was added to the mixture and heated for 3 h at 100° C. in a tightly closed pressure tube. After methanolysis, the samples were cooled on ice for 10 min, and 1 mL of ddH₂O per 2 mL of CHCl₃ (1:2 ratio) was added. The mixture was vortexed for 1 min, and the phases were separated by centrifugation at 4,500×g for 5 min, at 20° C. The organic phase was collected, neutralized with NaHCO₃ and dried over Na₂SO₄. The resulting mixture of 3-hydroxy-butanoyl methylester (3HBM) and further intracellular components in CHCl₃ were characterized and quantified by GC/FID (Agilent Technology GC system 7890A/5975 Inert XL EI/CI MSD with a triple axis detector, California, USA), using Poly[(R)-3-hydroxybutyric acid] purchased from Sigma-Aldrich (Sigma-Aldrich, Missouri, USA) as a reference.

The GC/FID was equipped with a DB-WAX (60 m×0.25 μm×0.5 μm, Agilent Technologies) column and ran a temperature profile starting at 50° C. for 1 min then increasing 15° C. min⁻¹ up to 240° C. for 5 min (total run time 18.6 min). The injection volume was 1 μL, and the flow rate was 1.7 mL min⁻¹.

Results and Discussion

Stage IA: CO₂ to Acetic Acid by A. woodii and the Influence of Pressure

Fermentation of A. woodii in a high-pressure stirred tank reactor was assessed using a H₂:CO₂ gas mixture of 85:15 at 2.0 bar and 5.5 bar to increase the solubility of gases with pressure to improve growth and acetic acid production. A sealed-high pressure reactor prevented the loss of gases introduced to the reactor. Gas consumption was recorded and energy efficiency calculated.

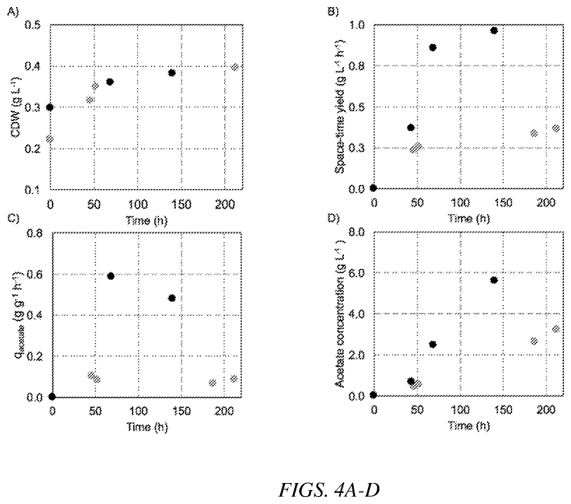

Growth and acetic acid production of A. woodii in ‘A2’ medium at pH 7.0 were monitored. At 2.0 bar pressure (H₂ partial pressure 1.7 bar, equal to 1.3 mM H₂), a total of 5.2 L gas was consumed. The cell dry weight (CDW) increased from 0.22 g L⁻¹ to 0.40 g L⁻¹ during the stationary phase (212 h of culturing). Acetic acid production was 3.20 g L⁻¹, with a space-time yield of 0.36 g L⁻¹ and a maximum cell-specific acetate formation rate (q_{acetate}) of 0.09 g_{AcOH} g_{CDW}⁻¹ h⁻¹ (2.1 g_{AcOH} g_{CDW}⁻¹ h⁻¹) during the exponential growth phase. The energy efficiency (η_{H2 to AcOH}) was 55% (FIGS. 4A-D).

The final acetate concentrations might seem low (3.20 g L⁻¹ after 212 h of culturing) compared to 59.2 g L⁻¹ after a processing time of approximately 77 h. These differences can be explained by the different approaches used. In our approach, the gas mixture (85 H₂:15 CO₂) was delivered by pressure regulation (when the gas is consumed the pressure drops, and the bpc 2 rebuilds the pressure), which prevents gas losses and allows a detailed measurement of gas consumption. Conversely, known methods using a gas mixture of H₂:CO₂:N₂ (60:25:15) with a constant flow rate of 30 L h⁻¹ at 1 bar pressure. This allows a higher gas-to-liquid mass transfer rate, resulting in a higher total yield, but also a loss of about 25% gas. At 5.5 bar pressure (H₂ partial pressure 4.67 bar, equal to 3.6 mM H₂), a total of 8.9 L gas was consumed. The CDW increased from 0.30 g L⁻¹ to 0.40 g L⁻¹ during the stationary phase (140 h of culturing). Acetate production (5.60 g L⁻¹), space-time yield (0.96 g L⁻¹ d⁻¹) and the maximum acetate formation rate, q_{acetate} (0.50 g_{AcOH} g_{CDW}⁻¹·h⁻¹) (12 g_{AcOH} g_{CDW}⁻¹·h⁻¹) were also substantially higher at 5.5 bar than at 2.2 bar. However, the energy efficiency (η_{H2 to AcOH}) at 5.5 bar pressure was lower (22%) (FIG. 4). Table 2 compares acetic acid production from CO₂ under different pressure conditions observed in these studies with the results of Lagoa-Costa et al.

TABLE 2
Bacterial growth under different pressures of CO₂:H₂ gas and different media.

| | Lagoa-Costa et al. | Instant Examples | | |
|---|---|---|---|---|
| Pressure (bar) | Atmospheric | 2.0 | 5.5 | 5.5 |
| Media | | A2 | A2 | A3 |
| Strain | C. autoethanogenum | | A. woodii | |
| Acetate production (g L⁻¹) | 2.66 | 3.20 | 5.60 | 4.50 |
| Space-time yield (g L⁻¹ d⁻¹) | 0.121 | 0.36 | 0.96 | 1.12 |
| q_{acetate} (g_{AcOH} g_{CDW}⁻¹ · h⁻¹) | 0.02 | 0.09 | 0.50 | 0.60 |
| Gas mixture | CO:CO₂:H₂:N₂ | | CO₂:H₂ (15:85) | |
| | (30:10:20:40) | | | |
| Gas utilized (L) | 5240 | 5.2 | 8.9 | 30 |
| Process time (h) | 523 | 212 | 140 | 96 |
| Energy efficiency (%) | N/A | 55 | 22 | 5.4 |
| (ηH2 to AcOH) | | | | |

Comparing the present examples to the approach of Lagoa-Costa et al., the results show that increasing the pressure of the gas mixture reduced the process time significantly, while increasing acetate production, space-time yield, and q_{acetate}. The use of pressure allows the moderate gas consumption without losses.

Moreover, a decrease in energy efficiency (η_{H2 to AcOH}) was observed when increasing the pressure from 2.0 bar to 5.5 bar, and at 5.5 bar the energy efficiency was further reduced with increasing nitrogen content. The reduced energy efficiency at 5.5 bar may result from the applied high partial pressure of CO₂ that causes a shift in the thermodynamic equilibrium, leading to the accumulation of CO in the carbonyl branch of the WLP (FIG. 2). Carbon monoxide inhibits the ability of A. woodii to use H₂ because the hydrogen-dependent carbon dioxide reductase (HDCR) is sensitive to CO, which results in the inhibition of acetate production from H₂:CO₂. A. woodii is known to generate CO when forming acetic acid at 1.7 bar of H₂:CO₂ (80:20) atmosphere in batch cultures. Additionally, the increased H₂ partial pressure in the gas phase or its accumulation in the fermentation medium may alter or inhibit the production of NADH, which consequentially redirects the carbon flow away from cell growth and acetic acid production by A. woodii in autotrophic batch processes.

Stage IB: Acetic Acid Production Rate in Enriched Medium at 5.5 Bar

The yeast extract content was increased from 4.0 g L⁻¹ to 6.0 g L⁻¹ (medium ‘A3’) to increase the energy efficiency (η_{H2 to AcOH}) at 5.5 bar, and prevent nutrient limitations and promote higher cell activity, to increase utilization of H₂ and CO₂.

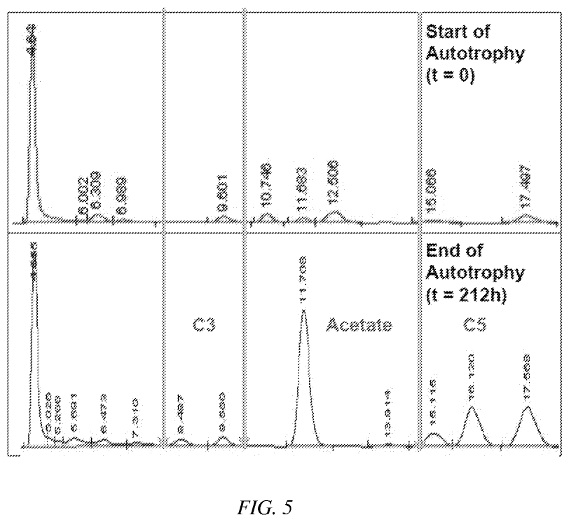

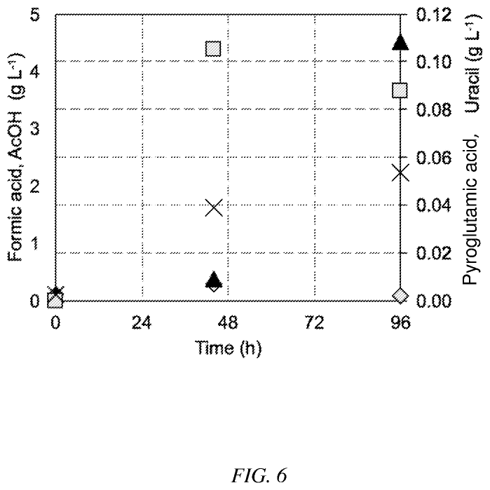

In the enriched medium (‘A3’), 30 L gas mixture (CO₂:H₂, 15:85) was consumed, and 4.50 g L⁻¹ acetic acid was produced in the stationary phase (96 h of cultivation). The space-time yield was 1.12 g L⁻¹·d⁻¹ and the maximum acetate formation rate, q_{acetate}, was 0.6 g_{AcOH} g_{CDW}⁻¹·⁻¹ compared to 0.96 g L⁻¹·d⁻¹ and g L⁻¹ and 0.50 g_{AcOH} g_{CDW}⁻¹·h⁻¹, respectively, with 4 g L⁻¹ yeast extract (medium ‘A2’) at 5.5 bar. However, the energy efficiency (η_{H2 to AcOH}) was 5.4% in medium ‘A3’ compared to 22% in medium ‘A2’ (Table 1). Also, substantially more gas (30 L compared to 8.9 L of CO₂:H₂ mixture) was consumed but less acetic acid was produced. Detailed analysis of the medium revealed that A. woodii produced other substances besides acetic acid, such as formic acid, uracil, pyroglutamate and traces of lactate (FIGS. 5 and 6) when grown in ‘A3’ comprising increased yeast extract relative to ‘A2’.

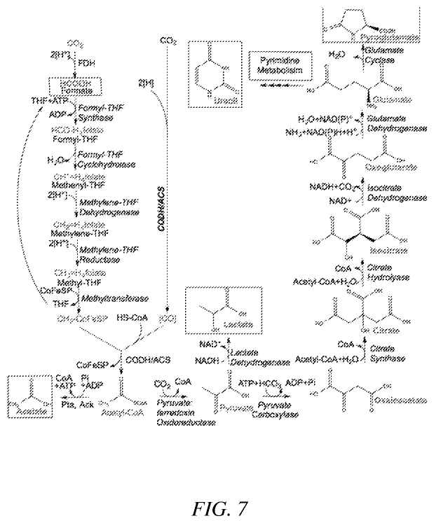

The differences in product formation in the enriched medium ‘A3’ likely resulted from the additional vitamins and nitrogen supplemented from the high content of yeast extract. These vitamins are essential cofactors for the WLP, and the additional nitrogen might promote the build-up of the nitrogen-containing products uracil and pyroglutamate. These results suggest that the high concentration of yeast extract in combination with the increased pressure led to a high production of NADH, which altered the carbon flow. The excess of NADH would then lead to the accumulation of acetyl-CoA in the WLP where acetyl-CoA is not only converted to acetic acid, but also to pyruvate. The results further suggest that the pyruvate is then utilized in the TCA cycle that produces more NADH, which, in turn, is used to synthesize uracil and pyroglutamic acid using the excess of nitrogen (FIG. 7).

Stage II: Conversion of Acetic Acid to PHB by R. eutropha H16

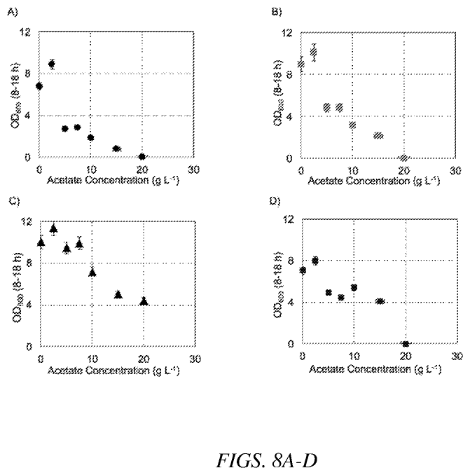

The effect of acetate concentration on aerobic cell growth was assessed to avoid the toxicity at higher concentrations of acetate and/or other byproducts. Therefore, R. eutropha H16 growth was investigated in parallel in 10 mL stirred tank bioreactors under varying concentrations of acetate and pH. R. eutropha H16 were grown in miniaturized stirred-tank reactors using ‘R2’ medium supplemented with 30 g L⁻¹ fructose at different pH levels of pH 6.5, pH 7.0, pH 7.5 and pH 8.0. After 8 h of growth, acetic acid was added to reach the following concentrations: 2.5 g L⁻¹, 5.0 g L⁻¹, 7.5 g L⁻¹, 10 g L⁻¹, 15 g L⁻¹, and 20 g L⁻¹. After 10 h (total 18 h) of batch cultivation, OD₆₀₀ was measured (FIGS. 8A-D).

At pH 7.5, no severe toxic effect was detected when the acetate concentration was up to 7.5 g L⁻¹, and the cell density increased to an OD₆₀₀ of 9.8. By increasing the acetate concentration up to 20 g L⁻¹ at pH 7.5, the growth increased to an OD₆₀₀ of 4.4 after 10 h. Despite the low increase, pH 7.5 allowed the bacteria to maintain better growth compared to pH 6.5, pH 7.0 and pH 8.5.

These results showed that the ideal process pH is 7.5 with a maximum acetic acid concentration of 7.5 g L⁻¹, where a consumption rate of 0.8 g L⁻¹ h⁻¹-OD₆₀₀ acetate was measured. However, at a lower pH of 6.5, a severe toxic effect of acetic acid started at relatively low concentrations of 5.0 g L⁻¹, which resulted in a decline in growth (FIGS. 8A-D). This severe effect is also visible at pH 7.0 with higher acetic acid concentrations (up to 10 g L⁻¹).

At pH 8.0, the decline in growth was visible at 15 g L⁻¹ acetic acid. Short-chain fatty acids (SCFA), such as acetic acid, tend to split into anions above their pKa. Acetic acid has a pKa of 4.76. Therefore, the higher the solution pH, the more acetic acid is split into anions. Although free anions can slowly enter the cytoplasm where they might adversely affect cell metabolism, cells can pump anions out of their cytoplasm. However, this mechanism of detoxification through anti-porters requires energy and results in a decreased growth rate and PHB production with increased anion concentrations.

At pH values below the pKa values of the SCFAs, SCFAs accumulate in their un-dissociated form, which can dissolve into the lipid bilayer of the cell membrane and act as uncoupling agents. Conversely, dissolved intact SCFAs allow protons to pass through the cell membrane by acting as proton carriers, resulting in an uncoupled electron transport from the ATP synthase. This electron transport and proton pumping continue at a rapid rate, but no proton gradient is generated, and ATP can no longer be synthesized. The lack of ATP is initially compensated by utilization of acetyl-CoA in the TCA cycle, which inhibits the production of PHB and results in activity inhibition and eventually cell death over time.

Under the conditions used, pH 7.5 appears to most favorably balance the different effects of intact or dissociated acetic acid. Notably, at alkaline conditions between pH 8.0 to 8.5, 99.9% of acetic acid remained in its dissociated form, but this pH range had an inhibitory effect on R. eutropha H16 growth. These findings underscore that pH control is important because it affects the ionization of the active components of microbial cells (enzymes, enzyme complexes, or other ionizable substrate receptors). For optimal growth and activity, these components must be in their appropriate ionic forms to bind their substrates. These results show the benefit of a slightly alkaline pH, while feeding acetic acid (20 g L⁻¹) with the highest cell yield at pH 7.5 with an OD₆₀₀ of 4.4 (after 10 h). In these studies, the comparatively high tolerance for acetic acid can be explained by the use of bioreactors. The controlled environment in bioreactors (e.g., no oxygen limitation and pH control) potentially allows improved cell growth because of the higher pumping capacity of the cells.

A two-stage bio logical process that allows the gas to liquid transfer (Bio-GTL) of CO₂ into the biopolymer polyhydroxybutyrate (PHB) has been demonstrated. First, acetic acid was produced by A. woodii by mixing CO₂:H₂ (15:85) gas under elevated pressure (≥2.0 bar) to increase H₂-solubility in water. Second, acetic acid was converted to PHB by R. eutropha H16. This Bio-GTL process achieved fixation and conversion using the same medium in both steps. Evaluation of the efficiencies and time-space yields under different bioprocessing conditions, including the partial pressures of H₂ and CO₂, acetic acid concentrations and pH levels in individual stirred tank batch reactors, show the laboratory scale two-stage bioprocessing method efficiently converts CO₂ into PHB with a 33.3% microbial cell content (percentage of the ratio of PHB concentration to cell concentration).

Bio-GTL Microbial Process to Convert the Produced Acetate from Co₂ Fermentation into PHB

The examples above focused on the use of two metabolically different strains (A. woodii and R. eutropha). Therefore, the development of a medium that supports both metabolic modes (gas fermentation and PHB production) was undertaken. Use of a single medium differs from the approach of Lagoa-Costa et al.

A medium that can first be used for A. woodii, and then be kept for R. eutropha after adjusting its pH was established. The medium resulting from stage 1 (‘A2’ under 2.0 bar) was used in stage 2, after pH adjustment. The medium was collected after A. woodii fermentation at 2.0 bar, containing 3.20 g L⁻¹ acetate. The pH of the medium was adjusted to 7.5 and inoculated with a R. eutropha H16 culture that was pre-grown to an OD₆₀₀ of 1.5 (0.27 g L⁻¹) in medium ‘R1’ and washed once with phosphate buffer (30 mM, pH 7.5). R. eutropha H16 grew to a final OD₆₀₀ of 9.3 (active biomass 0.41 g L⁻¹) after 5 h of culturing with an uptake of 3.0 g L⁻¹ acetate and a q_{acetate} 1.46 g_{AcOH} g·_{CDW}⁻¹ h⁻¹. PHB production was 0.5 g L⁻¹, and a q_{PHB} of 0.24 g_{PHB} g_{CDW}⁻¹ h⁻¹ and the PHB content (percentage of weight) in the microbial cell was 33.3%. The PHB content was defined as the percentage of the ratio of PHB concentration to cell concentration.

Despite the increase in biomass, due to the high nitrogen content from the acetogen media, R. eutropha had a higher q_{PHB} and a PHB content than the results reported by Lagoa-Costa et al. It appeared that the ‘A2’ medium successfully induced PHB production with a high PHB content after adjusting the pH to 7.5 (Table 3). Using a single media that only requires pH adjustment is the first step towards a continuous setup in which both the CO₂ based production of acetic acid and the conversion of acetic acid to PHB are connected to each other.

TABLE 3
Conceptual overview of the Bio-GTL microbial process as described
in the present examples in comparison to Lagoa-Costa et al.

| | Lagoa-Costa et al. | Present Examples |
|---|---|---|
| Microbial strain | C. autoethanogenum | R. eutropha H16 |
| Culturing | Fed-batch | Shaking flask |
| Total acetate uptake (g L⁻¹) | 4.0 (8 pulses, 0.5 g L⁻¹/pulse) | 3.0 with direct inoculation |
| Active biomass (mmol L⁻¹) | 0.01 | 16 |
| Growth (C_{CDW} mmol C_{AcOH} mmol⁻¹) | 0 | 0.32 |
| PHB storage (C_{PHB} mmol C_{AcOH} | 0.275 | 0.116 |
| mmol⁻¹) | | |
| q_{PHB} (C_{PHB} mmol/C_{CDW} mmol⁻¹ · h⁻¹) | 0.042 | 0.068 |
| PHB cell content (%) | 24 | 33.3 |

A Bio-GTL process to convert CO₂ into PHB in two stages was developed, using acetic acid as an intermediate. In the first stage, under high-pressure conditions, using a mixed CO₂:H₂ gas with a ratio of 15:85 CO₂ is converted to acetic acid by A. woodii. The bioreactor used in this example delivered the gas to the stirring tank only when pressure dropped as a result of microbial consumption. This system prevented loss in gas, which is advantageous compared to previous approaches, such as the method of Lagoa-Costa et al., where large gas volumes flow through the stirring tank. In the second step, acetic acid is converted to PHB by R. eutropha. A medium that can be used for both microbial processing stages by simply adjusting its pH was established. The resulting medium (‘A2’) was successful in inducing PHB production and accumulation with high percentage.

The results described above provide evidence to support upscaling the system of the instant examples (e.g., to a closed-loop continuous process). A closed loop two-stage process without the need for reconditioning the medium between stages would improve cost-effectiveness. For example, in a continuous process, the medium can be reused for several cycles to maximize production and reduced process costs. As a single closed loop, medium and nutrition input are minimal. The aerobic second stage would render the acetogens of the primary stage non-viable if the culture flowed directly into the aerobic fermenter. Harvesting the PHB-producing bacteria would require only one separator, to which renders this system a uniquely cost-effective approach relative to systems requiring multiple separation steps or medium formulations, or recovery of acetate from the medium.

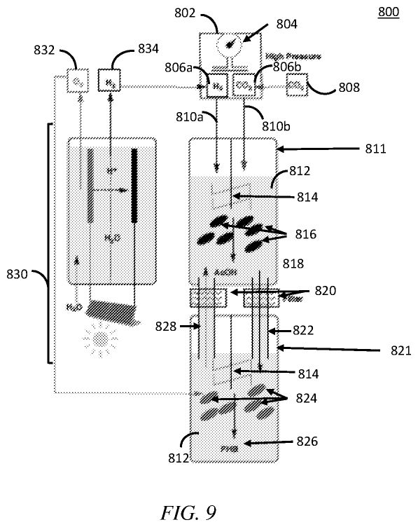

The sustainability of the scaled-up Bio-GLT process could be further improved with photo- or photoelectrically produced hydrogen. For example, a system coupled with photovoltaics to electrochemically produce H₂ for use with external CO₂ to drive a sustainable photovoltaic/Bio-GLT approach for the production of bioplastic is depicted in FIG. 9.

FIG. 9 illustrates an exemplary system for producing bioplastic from a gaseous substrate according to one or more embodiments of the present disclosure. System 800 is for the production of bioplastic products (e.g., PHB) from CO₂ and sustainably produced H₂. The system includes gaseous substrate supply unit 802 which is operably connected to pump 804 for pressurizing the gaseous substrate. Pump 804 can be a high-pressure pump configured to pressurize the gaseous substrate to a pressure in the range of more than about 2 bar (absolute). For example, a suitable high pressure pump can pressurize the gaseous substrate to a pressure of about 2-10, about 3-8, about 3-6, or about 2-5.5 bar (absolute). Pump 804 can be configured to record gas consumption and also to regulate the flow of the gaseous substrate to maintain a set pressure (e.g., by connection to a pressure regulating system, such as the bpc 2, Büchi pressflow controller; BÜCHIGLASUSTER®). The gaseous substrate can include gaseous electron source 806a and gaseous carbon source 806b. However, blended sources of gaseous substrate can be included in the system. The electron source includes H₂ gas, and the carbon source can include CO₂ gas, alone, or in admixture other gases (e.g., syngas or CO, CH₄, N₂, NH₃, H₂S, and other trace gases). Optionally, the gaseous carbon source can be a renewable source of gaseous carbon 808 such as a gaseous carbon stream obtained from steel mill waste gas, gasified solid waste, reformed biogas, pyrolyzed biomass. Gaseous electron source 806a and gaseous carbon source 806b are delivered by pump 804 through supply lines 810a and 810b, respectively, to primary bioreactor 811.

Primary bioreactor 811 can be configured for receiving the gaseous substrate comprising CO₂ and H₂ and enabling anaerobic fermentation of the gaseous substrate by a culture of an acetogenic microorganism at a pressure of at least 2 bar absolute to produce an acid. For example, the bioreactor can be an anaerobic fermenter including one or more vessels, beds, towers, or other suitable container configured to accommodate culture medium 812 (e.g., a liquid or flowable culture medium) and withstand high pressure (e.g., a sealable continuous stirred tank reactor). Primary bioreactor 811 may or may be configured to adapt gas flow within one or more of the vessels (e.g., the bioreactor may include or lack baffles). As shown, bioreactor 811 includes agitator 814, shown with a plurality of impellers, enabling high gas-liquid mass transfer coefficient in operation, which is connected to the bioreactor by a shaft. Agitator 814 can be a gas-inducing stirrer. A motor housing and motor that drives the stirring motion of the agitation shaft is present, although not shown. In one embodiment of the system, the primary bioreactor comprises a pressure regulating system enabling the pressure to be maintained during anaerobic fermentation.

When the system is in operation, culture medium 812 can be a liquid or flowable fermentation medium, containing an acetogenic microorganism 816 (e.g., a homoacetogen, as discussed above). In a continuous or semi-continuous manner, the acetogenic microorganisms convert the gaseous substrate to primary fermentation product 818 (e.g., one or more volatile fatty acids, such as acetate).

Primary bioreactor 811 can be configured to introduce a portion of culture medium 812 and primary fermentation product 818 into secondary bioreactor 821. As shown, primary bioreactor 811 is configured with conduit 822 configured to permit fermentation efflux to flow to the secondary bioreactor 821. Conduit 822 is shown with in-line separator 820. Separator 820 can be any suitable filtration device, including filters such as hollow fiber membranes or cross-flow membranes, configured to allow the primary fermentation product and culture medium to permeate. Alternatively, conduit 822 lacks a separator if the acetogen cells do not need to be removed from the culture medium flowing from the primary bioreactor to the secondary bioreactor, as described above.

Secondary bioreactor 821 can be configured to receive efflux from the primary bioreactor and to enable aerobic fermentation of the primary fermentation product to produce a secondary fermentation product. Secondary bioreactor 821 can be an aerobic fermenter including one or more tanks, vessels, towers or other containers adapted to accommodate culture medium 812. For example, secondary bioreactor 821 can be an aerated stirred tank reactor (e.g., a continuous stirred tank reactor) that includes agitator 814. The aeration can be nitrogen-limited aeration. Agitator 814 can be the same type of agitator as used in primary bioreactor 811 or can be adapted for faster agitation (e.g., based on target aeration rate). Secondary bioreactor 821 can be configured to adjust aeration rate and agitation speed to compensate for changes in dissolved oxygen content. Alternatively, a continuous flow reactor (e.g., a continuous plug flow tubular reactor) could be used as the secondary bioreactor in one or more embodiments. In operation, as shown, secondary bioreactor 821 includes culture medium 812 and a PHA-producing microorganism 824 (e.g., acetate-utilizing bacteria, as described above) capable of utilizing fermentation product 818 to produce secondary fermentation product 826 (e.g., one or more PHAs, such as PHB).

Secondary bioreactor 821 can be configured to monitor and adjust the pH of culture medium 812 for the growth of the PHA-producing microorganism. For example, in one or more embodiments, the secondary bioreactor is connected to a pH control system (not shown). A pH control system can include sensors for detecting pH (e.g., fluorimetric sensors) and/or optical density and a liquid handling system enabling the addition of acidic, alkaline or buffering substances in response to variances in the pH relative to a pre-set value (e.g., using dosing pumps) and growth rate.

Secondary bioreactor 821 can be configured to permit extraction of samples of PHA-producing microorganism 824 to determine the presence of secondary fermentation product 826 (e.g., with a port for sampling for external preparation, or an internal sample PHA staining and fluorescence detection system).

In a continuous or semi-continuous manner, secondary bioreactor 821 can be configured to permit recycling of culture medium 812 to primary bioreactor 811. As shown, culture medium 812 is passed through conduit 828 into primary bioreactor 811. Conduit 828 is shown with in-line separator 820. Separator 820 can be any suitable filtration device for separating the PHA-producing microorganism 824 from the effluent (media and fermentation product) 812, including filters such as hollow fiber membranes or cross-flow membranes. Separator 820 can facilitate separation of culture medium 812 and the PHA-producing microorganism 824. In addition, separator 820 can facilitate stripping dissolved oxygen from culture medium 812. For example, separator 820 can be configured to deaerate the separated culture medium by one or more of the devices and methods described above (e.g., deaerating the medium by sparging). Such devices and methods remove or reduce the dissolved oxygen content of media 812 being recycled via conduit 828 which may inhibit acetogenesis in primary bioreactor 811. In one embodiment, system 800 includes a recirculation pump to recycle culture medium 812 in a closed-loop via the conduits.

Secondary bioreactor 821 can be configured to permit harvesting of the PHA-producing microorganism 824 (e.g., bacteria) for extracting secondary fermentation product 826. Configurations for harvesting include sensors to determine optical density and/or PHA cell content, and ports or other access points enabling removal of the PHA-producing microorganisms for further processing. For example, secondary bioreactor 821 can be configured for separating the PHA-producing microorganism 824 (referred to as PHA-containing biomass) by being connected to a continuous centrifuge that separates culture medium from the cells. The centrifuge can be configured to enable the separated culture medium to flow into the primary bioreactor and to facilitate the transfer of the separated biomass to a lyophilizer for PHA extraction, as discussed above.

Bioreactors 811 and 821 can be configured to permit headspace gas to be sampled and measured. In one embodiment of the present disclosure, secondary bioreactor 821 is configured to prevent or limit evaporation of the medium during fermentation. For example, secondary bioreactor 821 can be adapted to have a saturated air inlet port in the headspace region of the bioreactor, which is connected to a source of sterile air saturated with water (not shown). The bioreactors can further include a cooling system.

Bioreactors 811 and 821 can be small-scale single-use bioreactors, or large-scale systems configured for clean-in-place and sterilize-in-place equipment, and/or include components and equipment capable of being cleaned and sterilized in place. For example, a clean in place or sterilize in place configured bioreactor can be made of highly polished stainless steel, with all or fewer than all vessels designed for high pressure (e.g., ≥2 bar (absolute)) and full vacuum to withstand steam-sterilization conditions. For example, primary bioreactor 811 can be a sealable vessel configured to be pressurized. Such systems are known in the art of industrial scale-fermentation and biopharmaceutical production.

Bioreactors 811 and 821 can be configured to permit monitoring, control and/or adjustment of one or more of the following conditions within the bioreactors: temperature, pH, foaming, optical density, and dissolved oxygen. Systems permitting monitoring, control, and/or adjustment can include one or more computer processors, a user interface for displaying data or for receiving user input (e.g., times for sampling, volumes of liquid to be added), and memory containing computer readable instructions to be executed by the processors and/or for storing data.

System 800 includes an external electron source 830. External electron source 830 includes a photovoltaic cell configured to provide electrochemically produced H⁺ and O₂, for use as sources of hydrogen gas 834 and oxygen gas 832. Hydrogen gas source 834 operably connected to gaseous substrate supply 802. Oxygen gas source 832 can be configured to enable aerobic fermentation in bioreactor 821.

### Example 2

### Hydrogenotrophic Conversion of Co₂ into Bioplastic Via Two-Stage Bioprocess for a Sustainable Future

Atmospheric CO₂ concentration is rising to an alarming level (400 ppm) influencing global warming and the diversity of species. Slow degrading plastics represent a challenge for reducing global pollution. This example focuses on developing a sustainable bioprocess to capture and convert CO₂ into a biodegradable bioplastic. These results support the development of a continuous process that recycles the medium after PHB production, which further increases the sustainability of the process.

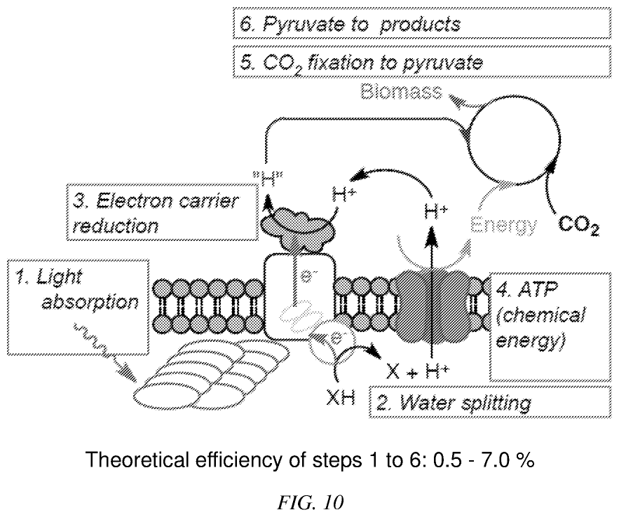

Photosynthesis is the largest biological route for CO₂ capture and conversion. The efficiency of photosynthesis (sun to bio-product energy) can be calculated based on the efficiency of specific points in the pathway to a theoretical value of 0.5-7.0%, however, 0.1-1.0% efficiency is achieved in nature (FIG. 10).

The Bio-GTL system mimics photosynthesis using a two-stage bioprocess including capturing CO₂ via autotrophic fermentation using A. woodii to produce acetate from a syngas substrate (CO₂ and H₂) and converting the acetate to higher carbon products (e.g., as a substrate in the production of biodegradable bioplastic such as poly(3-hydroxybutyrate) (PHB) in Ralstonia eutropha H16). Acetate is the major product of fermentation, with products such as formic acid, uracil, pyroglutamate and traces of lactate also identified (FIG. 5-6).

Other embodiments of the present disclosure are possible. Although the description above contains much specificity, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with or substituted for one another in order to form various embodiments. Thus, it is intended that the scope of at least some of the present disclosure should not be limited by the particular disclosed embodiments described above.

Thus, the scope of this disclosure should be determined by the appended claims and their legal equivalents. Therefore, it will be appreciated that the scope of the present disclosure fully encompasses other embodiments which may become obvious to those skilled in the art, and that the scope of the present disclosure is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims.

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to explain the principles of the disclosure best and its practical application to thereby enable others skilled in the art to best utilize the disclosure in various embodiments and with various modifications as are suited to the particular use contemplated. It is intended that the scope of the disclosure be defined by the claims appended hereto

Various examples have been described. These and other examples are within the scope of the following claims.

1. A method of producing bioplastic from a gaseous substrate comprising
a carbon source, the method comprising:
introducing a gaseous substrate comprising CO₂ and H₂ into a primary bioreactor comprising a culture of an acetogenic microorganism in a culture medium;
anaerobically fermenting the gaseous substrate in the primary bioreactor at a pressure of at least 2 bar absolute to produce an acid-containing culture medium;
introducing at least a portion of the acid-containing culture medium to a secondary bioreactor;
inoculating the secondary bioreactor with a culture of a polyhydroxyalkanoate (PHA)-producing microorganism, and
aerobically fermenting the acid in culture medium to produce a PHA.

2. The method of claim 1, wherein the CO₂ and H₂ are present in the gaseous substrate at a ratio of H₂:CO₂ within a range of about 75:25 to about 95:5.

3. The method of claim 1, wherein the H₂ partial pressure in the primary bioreactor is within a range of 1.7 bar to about 5 bar absolute.

4. The method of claim 1, wherein after consumption of a portion of the gaseous substrate by the acetogenic microorganism, the method further comprises introducing sufficient additional gaseous substrate to maintain the pressure in the primary bioreactor at or above 2 bar absolute.

5. The method of claim 1, wherein the acetogenic microorganism is selected from the group consisting of Acetobacterium, Clostridium, Eubacterium, Bacteroides, Sporomusa, Acetogenium, and Morera.

6. The method of claim 1, wherein the PHA-producing microorganism is selected from the group consisting of Cupriavidus necator H16, Pseudomonas putida mt-2, Bacillus spp. type strains, Corynebacterium glutamicum, Corynebacterium hydrocarboxydans, Nocardia lucida, and Rhodococcus sp.

7. The method of claim 1, further comprising adjusting the pH of the portion of acid-containing culture medium in the secondary bioreactor before inoculating with the culture of the PHA-producing microorganism.

8. The method of claim 7, wherein the pH is adjusted within a range of about 7 to about 8.

9. The method of claim 1, wherein introducing the portion of the acid-containing medium is performed when the acid concentration in the primary bioreactor is within a range of about 2.5 g L⁻¹ to about 20 g L⁻¹ culture medium.

10. The method of claim 1, wherein introducing the portion of the acid-containing culture medium comprises separating the acetogenic microorganism from the acid-containing culture medium.

11. The method of claim 1, further comprising obtaining the gaseous substrate from a renewable resource.

12. The method of claim 1, comprising obtaining the H₂ of the gaseous substrate by photovoltaic electrolysis, photoelectrochemical water splitting, or biomass gasification.

13. The method of claim 1, further comprising separating the PHA-producing microorganism in the secondary bioreactor to produce a separated culture medium and introducing the separated culture medium to the primary bioreactor.

14. The method of claim 1, further comprising extracting the PHA from the PHA-producing microorganism.

15. The method of claim 1, further comprising oxygenating the culture medium in the secondary bioreactor.

16. The method of claim 15, wherein oxygenating comprises introducing oxygen obtained from a renewable resource into the secondary bioreactor.

17. The method of claim 1, wherein the primary and secondary bioreactors are connected.

18. A system for producing bioplastic from a gaseous substrate comprising
a carbon source, the system comprising:
a primary bioreactor and a secondary bioreactor,
wherein the primary bioreactor is configured for receiving a gaseous substrate comprising CO₂ and H₂ and enabling anaerobic fermentation of the gaseous substrate at a pressure of at least 2 bar absolute for the production of an acid-containing culture medium by an acetogenic microorganism;
wherein the secondary bioreactor is configured for receiving at least a portion of the acid-containing culture medium from the primary bioreactor and enabling aerobic fermentation of the acid for the production of a polyhydroxyalkanoate (PHA) by a PHA-producing microorganism.

19. The system of claim 18, wherein the primary bioreactor comprises a pressure regulating system enabling the pressure to be maintained during anaerobic fermentation.

20. A culture medium for the production of a polyhydroxyalkanoate (PHA) by a PHA-producing microorganism, the culture medium made by a process comprising:
introducing a gaseous substrate comprising CO₂ and H₂ into a primary bioreactor comprising a culture of an acetogenic microorganism in an anaerobic culture medium;
anaerobically fermenting the gaseous substrate in the primary bioreactor at a pressure of at least 2 bar absolute to produce an acid-containing culture medium;
introducing at least a portion of the acid-containing culture medium to a secondary bioreactor; and
adjusting the pH of the acid-containing culture medium for growing a culture of a PHA-producing microorganism and producing a PHA by aerobic fermentation.
